(19) United States
(12) Patent Application Publication
(10) Pub. No.: US 2011/0077263 A1
(43) Pub. Date: Mar. 31, 2011

(54) Methods and Compositions of Toll-Like Receptor (TLR) Agonists

(75) Inventors: W. Martin Kast, Los Angeles, CA (US); Diane M. Da Silva, Los Angeles, CA (US)

(73) Assignee: University of Southern California

(21) Appl. No.: 12/892,875

(22) Filed: Sep. 28, 2010

Related U.S. Application Data
(60) Provisional application No. 61/246,881, filed on Sep. 29, 2009.

(51) Int. Cl. A61K 31/437; C12N 5/071; C12Q 1/70; A61P 35/00

(52) U.S. Cl. 514/293; 435/375; 435/5

(57) ABSTRACT

There is provided a method of activating a Langerhans cell (LC) exposed to a human papillomavirus (HPV) to induce a HPV-specific immune response, by administering to a subject an effective amount of a toll-like receptor (TLR) agonist, thereby activating the LC exposed to the HPV to induce the HPV-specific immune response.

### CROSS REFERENCE TO RELATED APPLICATION

This application claims the benefit under 35 U.S.C. §119(e) of U.S. Provisional Ser. No. 61/246,881, filed Sep. 29, 2009, the content of which is incorporated by reference in its entirety.

### STATEMENT OF GOVERNMENT SUPPORT

This invention was made with government support under Grant No. RO1 CA74397 awarded by the National Institutes of Health. The U.S. Government has certain rights in the invention.

### FIELD OF THE INVENTION

The invention relates to compositions, formulations, kits, assays, and methods for activation of a Langerhans cell (LC) exposed to a human papillomavirus (HPV) and a treatment of acute or persistent HPV infection or pre-cancerous lesions induced by acute or persistent HPV infection by using one or more of toll-like receptor (TLR) agonists.

### BACKGROUND

High-risk human papillomaviruses (HPV) 3 have been linked to the generation of cervical cancer (Walboomers et al. J. Pathol. 182: 12-19 (1999) and zur Hausen (1991) Virology 184:9-13). Cervical cancer may be the second most common cancer among women worldwide, killing approximately one-quarter of a million women each year. The majority of women infected with HPV may clear the virus; however, the average time for clearance is close to 1 year. Conversely, ˜15% of women that have high-risk HPV infections may not initiate an effective immune response against HPV and persistence of high-risk HPV infection may be a major risk factor in the development of cervical cancer.

Langerhans cells (LC) are the resident antigen-presenting cells (APCs) at the site of infection and therefore are responsible for initiating an immune response against HPV16. However, LC exposed to HPV16 do not induce a specific T cell immune response, which leads to the immune evasion of HPV16. The slow clearance rate and lack of an effective immune response indicates that HPV is escaping immune detection.

Individuals infected with human immunodeficiency virus (HIV-1) have a higher prevalence of human papillomavirus (HPV) infection and a 5-fold increased incidence of HPV-related cancers due to impaired T cell function. Cervical and anal cancers are caused by persistent infection with high-risk oncogenic HPV genotypes. Currently, there is no treatment for persistent HPV infection. Because HPV-related cancers are so prevalent in HIV-infected individuals, there is a need to develop strategies to reduce the risk and prevent the development of HPV infection and HPV-associated malignancies.

### SUMMARY OF THE INVENTION

Langerhans cells (LC) which are the resident antigen-presenting cells (APCs) at the site of the HPV infection and are responsible for initiating an immune response against HPV16, do not induce a specific T cell immune response after exposure to HPV, which leads to the immune evasion of HPV16.

This disclosure provides unexpected and surprising results in that TLR agonists can activate LC cells that have been exposed to HPV in patients infected with HPV or co-infected with HPV and HIV, to induce HPV specific immune response. It is shown herein that the interaction of HPV16 with LC inhibits their maturation, preventing the induction of HPV-specific T cell responses despite the presentation of viral antigens by LC (Fahey et al. (2009) The Journal Of Immunology 182:2919-2928). The treatment with TLR agonists induces HPV-exposed LC to activate HPV-specific T cells, and thereby clear the acute or persistent HPV infection and prevent the onset of cancer or reduce the likelihood of the development of the precancerous lesions, such as cervical cancer in women and anal cancer in men. The identification of TLR agonists that reverse HPV immune escape can lead to clinical trials for the treatment of persistent HPV infections and HPV-induced lesions in both the general population and in HIV-infected individuals.

In one aspect of the disclosure, there is provided a method for activating a Langerhans cell (LC) exposed to a human papillomavirus (HPV), comprising, or alternatively consisting essentially of, or yet further consisting of, contacting the LC with an effective amount of a toll-like receptor (TLR) agonist, thereby activating the LC. The contacting can be in vitro, ex vivo or in vivo.

In one aspect of the disclosure, there is provided a method to reverse human papillomavirus (HPV) immune escape in a subject, comprising, or alternatively consisting essentially of, or yet further consisting of, administering to a subject an effective amount of a toll-like receptor (TLR) agonist, thereby reversing the HPV immune escape in the subject.

In one aspect of the disclosure, there is provided a method for treating human papillomavirus (HPV) infection in a subject, comprising, or alternatively consisting essentially of, or yet further consisting of, administering to the subject an effective amount of a toll-like receptor (TLR) agonist, thereby treating the HPV infection in the subject. In one aspect, the HPV infection is an acute or persistent HPV infection.

In one aspect, the method further comprises administering to the subject an effective amount of an inflammatory agent, an analgesic, or an anti-human immunodeficiency virus (HIV) agent. The anti-HIV agent, in some aspects, is selected from the group of nucleoside and nucleotide reverse transcriptase (RT) inhibitors; non-nucleoside reverse transcriptase inhibitors; protease inhibitors (PIs); viral absorption inhibitors; or viral coreceptor agonists. In some aspects, the analgesic is selected from the group of paracetamol, non-steroidal anti-inflammatory drug, COX-2 inhibitor, opiate or morphinomimetic.

In one aspect of the disclosure, there is provided a method of treating pre-cancerous lesions induced by HPV infection in a subject, comprising, or alternatively consisting essentially of, or yet further consisting of, administering to a subject an effective amount of a toll-like receptor (TLR) agonist, thereby treating the pre-cancerous lesions induced by HPV infection in the subject.

In another aspect of the disclosure, there is provided an in vitro method for activating a Langerhans cell (LC) exposed to a human papillomavirus (HPV) to induce a HPV-specific immune response, comprising, or alternatively consisting essentially of, or yet further consisting of, contacting the LC exposed to HPV with a composition comprising an effective amount of a toll-like receptor (TLR) agonist, and assaying the induced HPV-specific immune response.

In another aspect of the disclosure, there is provided a method for screening of toll-like receptor (TLR) agonist for the treatment of an acute or persistent human papillomavirus (HPV) infection or a pre-cancerous lesion induced by HPV infection, comprising, or alternatively consisting essentially of, or yet further consisting of:

(i) administering a toll-like receptor (TLR) agonist to a test sample containing a Langerhans cell (LC) exposed to a human papillomavirus (HPV) to induce a HPV-specific immune response;

(ii) determining a level of HPV-specific immune response or determining a presence or absence of HPV DNA sequences and/or viral replication; and

thereby screening for TLR agonist for the treatment of an acute or persistent human papillomavirus (HPV) infection or a pre-cancerous lesion induced by HPV infection.

In yet another aspect of the disclosure, there is provided a method of treating an acute or persistent human papillomavirus (HPV) infection or a pre-cancerous lesion induced by HPV infection in a subject, comprising, or alternatively consisting essentially of, or yet further consisting of, administering to a subject an effective amount of a toll-like receptor (TLR) agonist in combination with another therapy selected from inflammatory agent, analgesic, or anti-human immunodeficiency virus (HIV) agent, thereby treating the an acute or persistent human papillomavirus (HPV) infection or a pre-cancerous lesion induced by HPV infection in the subject.

In one aspect of the disclosure, there is provided a pharmaceutical formulation for a treatment of an acute or persistent human papillomavirus (HPV) infection or a pre-cancerous lesion induced by HPV infection in a subject, comprising, or alternatively consisting essentially of, or yet further consisting of, an effective amount of a toll-like receptor (TLR) agonist and a pharmaceutically acceptable carrier.

Further provided is use of the above-mentioned compositions in the manufacture of a medicament for reversing HPV immune escape or inhibiting HPV infection in a LC, tissue containing LC or a subject having or at risk of HPV infection or treating pre-cancerous lesion induced by HPV infection in a subject. The medicaments may further comprise additional pharmaceuticals or agents that induce a localized immune response. These may be combined with pharmaceutically acceptable carriers that are suitable for the modes of administration.

In one aspect of the disclosure, there is provided a kit for a treatment of an acute or persistent human papillomavirus (HPV) infection or a pre-cancerous lesion induced by HPV infection in a subject, comprising, or alternatively consisting essentially of, or yet further consisting of: an effective amount of a toll-like receptor (TLR) agonist.

### BRIEF DESCRIPTION OF THE DRAWINGS

This invention is further described in detail in the following figures:

FIG. 1 illustrates characterization of monocyte-derived LC. In FIG. 1A, Monocyte derived LC were stained with either antilangerin, anti-CD1a, anti-E-cadherin (black histograms) or isotype-matched negative controls (gray histograms). The cells were analyzed by flow cytometry. LC generated from monocytes express langerin, CD1a, and E-cadherin. In FIG. 1B, Monocyte-derived LC were left untreated or exposed to HPV16 VLP and then permeabilized, fixed, and stained with either anti-TLR7, anti-TLR8 Abs (black histograms), or isotype-matched negative controls (gray histograms). The cells were analyzed by flow cytometry. Immature LC and LC exposed to HPV16 VLP express similar levels of TLR7 and TLR8. One representative experiment of three is shown.

FIG. 2 illustrates a differential expression of surface markers on DC and LC stimulated with imidazoquinolines. In FIG. 2A, DC were left untreated, treated with LPS, or treated with each of the imidazoquinolines. The cells were analyzed by flow cytometry for the expression of MHC class I and II molecules, CD80, and CD86. Surface markers are up-regulated when treated with 3M-002, imiquimod, resiquimod, and 3M-031. These data are represented by fold increase in surface marker expression, which are based on mean fluorescence intensity. The mean±SEM of four separate experiments is presented (*, p<0.05). In FIG. 2B, LC were left untreated, stimulated with LPS, exposed to HPV16 VLP, treated with each of the imidazoquinolines, or exposed to HPV16 VLP and subsequently treated with each of the imidazoquinolines. After the final incubation, the cells were analyzed by flow cytometry for the expression of MHC class I and II molecules, CD80, and CD86. 3M-002 and resiquimod induced the up-regulation of surface markers on LC and LC exposed to HPV16 VLP. These data are represented by fold increase in surface marker expression, which are based on mean fluorescence intensity. The mean±SEM of four separate experiments is presented (*, p<0.05; **, p<0.01; and ***, p<0.001).

FIG. 3 illustrates that 3M-002 and resiquimod highly induce the secretion of Th1-associated cytokines and chemokines by LC previously incubated with or without HPV16 VLP. Supernatants collected from untreated LC, LC exposed to HPV16 VLP, LC treated with each of the imidaziquinolines, or LC exposed to HPV16 VLP and then treated with imidaziquinolines were analyzed in triplicate for the presence of cytokines and chemokines Cytokine and chemokine levels were quantified using a human cytokine LINCOplex assay. These data are expressed as the mean concentration with error bars representing the SD (*, p<0.05 and **, p<0.001). The experiment was repeated three times and yielded similar results.

FIG. 4 illustrates that 3M-002 and resiquimod induce the up-regulation of CCR7 and migration of LC exposed to HPV16 VLP toward CCL21. LC were left untreated, stimulated by LPS, exposed to HPV16 VLP, or exposed to HPV16 VLP and subsequently treated with each of the imidaziquinolines. After the final incubation, LC were either: FIG. 4A, harvested and analyzed for the expression of CCR7 (black line) by flow cytometry (gray line is the isotype control Ab) or FIG. 4B, used in a migration assay. The mean±SEM of three separate experiments is presented (***, p<0.001).

FIG. 5 illustrates that 3M-002 and resiquimod induce an HPV16 epitope-specific CD8⁺ T cell immune response through the activation of LC exposed to HPV16 cVLP. LC were incubated with medium alone or with HPV16 cVLP and each of the imidazoquinolines. In control experiments, LC were treated with each of the imidazoquinolines and pulsed with a HPV16-E7-derived HLA-A*0201-restricted CTL epitope. The treated LC were incubated with autologous CD8⁺ lymphocytes and restimulated twice. Responder cells were analyzed in triplicate for IFN-γ production in an ELISPOT assay against the E786-93 peptide. The number of spots in each well was counted and averaged. These data are expressed as the mean±SEM (*, p<0.05 and **, p<0.01). The experiment was repeated three times using two independent HLA-A*0201-positive donors and yielded similar results.

FIG. 6 illustrates LC treated with TLR 3/7/8 agonists after HPV16 L1L2 VLP exposure secrete IL-12 (Example 6).

FIG. 7 shows that LC treated with Poly-ICR after HPV16 L1L2 VLP exposure upregulate MHC and co-stimulatory molecules. LC were left untreated or exposed to HPV16 L1L2 VLP for 6 h at 37° C. Subsequently, the cells were treated with 5 μg/mL Poly-ICR or 20 μg/mL CD40L for 48 h at 37° C. After the final incubation the cells were stained with anti-human HLA-DPDQ (MHC class II), HLA-ABC (MHC Class I), CD40, CD80, CD83, and CD86 antibodies and analyzed by flow cytometry. Data represent fold increase in expression of each surface molecule (±SEM) of three individual donors relative to untreated LC based on the MFI. ***P<0.001, **P<0.01, *P<0.05 compared to both untreated LC and LC exposed to HPV16 VLP.

FIG. 8 shows Secretion of inflammatory cytokines and chemokines by LC treated with Poly-ICR after HPV16 L1L2 VLP exposure. LC were left untreated or exposed to HPV16 L1L2 VLP for 6 h at 37° C. Subsequently, the cells were treated with 5 μg/mL Poly-ICR or 20 μg/mL CD40L for 48 h at 37° C. Cell supernatants were analyzed for a panel of cytokines and chemokines using a Bio-plex suspension bead ELISA (BioRad, Hercules, Calif.). Shown is a representative cytokine profile of Poly-ICR treated LC from one healthy donor of three donors tested.

FIG. 9 demonstrates in vitro migration of LC treated with Poly-ICR after HPV16 L1L2 VLP exposure. LC were left untreated or exposed to HPV16 L1L2 VLP for 6 h at 37° C. Subsequently, the cells were treated with 5 μg/mL Poly-ICR or 20 μg/mL CD40L for 48 h at 37° C. Cells were analyzed for migration through a 5 μm transwell insert to medium or medium supplemented with 250 ng/mL CCL21/SLC. After 4 h, cells migrating to the lower chamber containing chemokine were counted. Shown is the mean migration index calculated as the number of cells migrating to CCL21 over spontaneous migration (±SEM) of four individual donors relative to untreated LC. **P<0.01, *P<0.05 compared to untreated LC.

FIG. 10 shows proliferation of allogeneic T cells by HPV16-exposed LC treated with Poly-ICR. LC were left untreated or exposed to HPV16 L1L2 VLP for 6 h at 37° C. Subsequently, the cells were treated with 5 μg/mL Poly-ICR 48 h at 37° C. LC were co-cultured with purified MHC-mismatched T cells from a healthy donor for 6 days in triplicate wells. 3H-thymidine was added during the last 8 hours of culture. Proliferation indices were calculated as [mean radioactive counts per minute (cpm) experimental/mean cpm of T cells alone]. Shown is the mean proliferation index (±SEM) of three individual donors. *P<0.05 compared to untreated LC.

FIG. 11 shows results of MHC tetramer binding analysis of HPV16-specific T cells after in vitro immunization with Poly-ICR treated LC after HPV16 L1L2E7 VLP exposure. MHC tetramer analysis of HPV16 L1L2-E7 cVLP loaded LC treated with Poly-ICR or resiquimod against the known E7-derived HLA-A*0201-restricted CTL epitope (E7₈₆₋₉₃, TLGIVCPI). CD8+ T cells were co-cultured with treated or untreated autologous LC for 4 weeks with weekly restimulations. LC were loaded with HPV16 L1L2-E7 chimeric VLP, then treated with Poly-ICR or Resiquimod (positive control). One week after the final restimulation, T cells were collected and stained with fluorescent labeled MHC class I tetramers specific for T cells recognizing the E⁷₈₆₋₉₃ peptide epitope and antibodies to CD8 and CD3 T cell markers, then analyzed by flow cytometry. Shown is the % E7₈₆₋₉₃ tetramer positive CD8+ T cells after a four week in vitro co-culture with treated LC. Results are representative of three individual healthy donors tested.

FIG. 12 shows activation of IFNγ secreting HPV16-specific T cells by LC treated with Poly-ICR after HPV16 L1L2E7 VLP exposure. IFNγ Elispot analysis after in vitro immunization of naïve T cells from a healthy donor against the HLA-A*0201-restricted CTL epitope E7₈₆₋₉₃. CD8+ T cells were cultured as described in FIG. 5, collected and tested for IFNγ secretion in response to E7₈₆₋₉₃ peptide stimulation. The number of spots representing IFNγ secreting cells were counted and averaged over 8 wells, subtracting background values (no peptide stimulation). The experiments were performed with LC from three HLA-A*0201⁺ healthy donors. Shown is a representative example of the number of IFNγ-secreting HPV-specific CD8+ T cells (±SEM) of one individual donor of three healthy donors tested. *P<0.05 compared to untreated LC.

### DETAILED DESCRIPTION OF THE INVENTION

Throughout this application, the text refers to various embodiments of the present compounds, compositions, and methods. The various embodiments described are meant to provide a variety of illustrative examples and should not be construed as descriptions of alternative species. Rather it should be noted that the descriptions of various embodiments provided herein may be of overlapping scope. The embodiments discussed herein are merely illustrative and are not meant to limit the scope of the present disclosure.

Also throughout this disclosure, various publications, patents and published patent specifications are referenced by an identifying citation. The disclosures of these publications, patents and published patent specifications are hereby incorporated by reference into the present disclosure in their entirety to more fully describe the state of the art to which this diclosure pertains.

### A. Definitions

The practice of the present disclosure will employ, unless otherwise indicated, conventional techniques of organic chemistry, pharmacology, immunology, molecular biology, microbiology, cell biology and recombinant DNA, which are within the skill of the art. See, e.g., Sambrook, Fritsch and Maniatis, MOLECULAR CLONING: A LABORATORY MANUAL, 2^{nd} edition (1989); CURRENT PROTOCOLS IN MOLECULAR BIOLOGY (F. M. Ausubel, et al. eds., (1987)); the series METHODS IN ENZYMOLOGY (Academic Press, Inc.): PCR 2: A PRACTICAL APPROACH (M. J. MacPherson, B. D. Hames and G. R. Taylor eds. (1995)), Harlow and Lane, eds. (1988) ANTIBODIES, A LABORATORY MANUAL, and ANIMAL CELL CULTURE (R. I. Freshney, ed. (1987)).

As used in the specification and claims, the singular form “a,” “an” and “the” include plural references unless the context clearly dictates otherwise. For example, the term “a cell” includes a plurality of cells, including mixtures thereof.

As used herein, the term “comprising” is intended to mean that the compositions and methods include the recited elements, but not excluding others. “Consisting essentially of” when used to define compositions and methods, shall mean excluding other elements of any essential significance to the combination. Thus, a composition consisting essentially of the elements as defined herein would not exclude trace contaminants from the isolation and purification method and pharmaceutically acceptable carriers, such as phosphate buffered saline, preservatives, and the like. “Consisting of” shall mean excluding more than trace elements of other ingredients. Embodiments defined by each of these transition terms are within the scope of this disclosure.

All numerical designations, e.g., pH, temperature, time, concentration, and molecular weight, including ranges, are approximations which are varied (+) or (−) by increments of 0.1. It is to be understood, although not always explicitly stated that all numerical designations are preceded by the term “about.” It also is to be understood, although not always explicitly stated, that the reagents described herein are merely exemplary and that equivalents of such are known in the art.

An “agonist”, as used herein, refers to a drug or other chemical that can bind a receptor on a cell to produce a physiologic reaction typical of a naturally occurring substance. The efficacy of an agonist may be positive, causing an increase in the receptor's activity.

“Administration”, as used herein, refers to the delivery of a medication, such as the agent of the disclosure, which reverses HPV immune escape or treats HPV infection or treats pre-cancerous lesions induced by HPV infection, to an appropriate location of the subject, where a therapeutic effect is achieved. Non-limiting examples include oral dosing, intracutaneous injection, direct application to target area proximal areas on the skin, or applied on a patch. Various physical and/or mechanical technologies are available to permit the sustained or immediate topical or transdermal administration of macromolecules (such as, peptides).

A “composition” is intended to mean a combination of active agent, cell or population of cells and another compound or composition, inert (for example, a detectable agent or label or biocompatible scaffold) or active, such as a growth and/or differentiation factor.

A “control” is an alternative subject or sample used in an experiment for comparison purpose. A control can be “positive” or “negative”. For example, where the purpose of the experiment is to determine a correlation of an altered level of HPV specific immune response, it is generally preferable to use a positive control (a sample from a subject, carrying such alteration and exhibiting the desired immune response), and a negative control (a subject or a sample from a subject lacking the immune response). Alternatively, a positive control is an agent exhibiting a desired biological response and a negative control is one that is known not to exhibit the desired biological response.

As used herein, “effective amount” is an amount sufficient to effect beneficial or desired results. An effective amount can be administered in one or more administrations, applications or dosages. Such delivery is dependent on a number of variables including the time period for which the individual dosage unit is to be used, the bioavailability of the therapeutic agent, the route of administration, etc. It is understood, however, that specific dose levels of the TLR agonists and other agents in the present disclosure for any particular subject depends upon a variety of factors including the activity of the specific compound employed, bioavailability of the compound, the route of administration, the age of the animal and its body weight, general health, sex, the diet of the animal, the time of administration, the rate of excretion, the drug combination, and the severity of the particular disorder being treated and form of administration. Treatment dosages generally may be titrated to optimize safety and efficacy. Typically, dosage-effect relationships from in vitro and/or in vivo tests initially can provide useful guidance on the proper doses for patient administration. Studies in animal models generally may be used for guidance regarding effective dosages for treatment of diseases such as cancer. In general, one will desire to administer an amount of the compound that is effective to achieve a serum level commensurate with the concentrations found to be effective in vitro. These considerations, as well as effective formulations and administration procedures are well known in the art and are described in standard textbooks.

As used herein, “HPV-specific immune response” refers to activation of NF-κB and other transcription factors in LC, phenotypic and functional changes including up-regulation of co-stimulatory molecules CD80 and CD86, MHC class I and II, chemokine receptors such as CCR7, secretion of cytokines and chemokines, and migration to regional lymph nodes where T cell activation takes place. The cytokines and chemokines include, but are not limited to, TNF-α, IL-6, IL-8, IL-12, and IFN-inducible protein 10 (IP 10), produced by dendritic cells (DC) and macrophages. In some embodiments, the “HPV-specific immune response” refers to the activation and expansion of HPV-specific T lymphocytes that recognize HPV-derived peptides in the context of MHC class I and class II molecules. The functions of HPV-specific T cells include, but are not limited to, recognition and elimination of HPV virus-infected cells through contact-dependent or contact-independent mechanisms, secretion of cytokines, and formation of long-lived memory T lymphocytes with the capacity for rapid proliferation and function upon secondary encounter with HPV antigens.

A “pharmaceutical composition” is intended to include the combination of an active agent with a carrier, inert or active such as a biocompatible scaffold, making the composition suitable for diagnostic or therapeutic use in vitro, in vivo or ex vivo.

As used herein, “pharmaceutically acceptable carrier” encompasses any of the standard pharmaceutical carriers, such as a phosphate buffered saline solution, water, and emulsions, such as an oil/water or water/oil emulsion, and various types of wetting agents. The compositions also can include stabilizers and preservatives. For examples of carriers, stabilizers and adjuvants, see Martin, Remington's Pharm. Sci., 15th Ed. (Mack Publ. Co., Easton (1975)). The term includes carriers that facilitate controlled release of the active agent as well as immediate release.

For topical use, the pharmaceutically acceptable carrier is suitable for manufacture of creams, ointments, jellies, gels, solutions, suspensions, etc. Such carriers are conventional in the art, e.g., for topical administration with polyethylene glycol (PEG) or carboxymethylcellulose. These formulations may optionally comprise additional pharmaceutically acceptable ingredients such as diluents, stabilizers, and/or adjuvants.

As used herein, “pre-cancerous lesions induced by HPV infection” refers to malignant and benign epithelial proliferative lesions related to diseases such as carcinoma of the cervix of the uterus (cervical carcinoma) and other anogenital cancers such as anal cancer, vaginal cancer, vulvar cancer, penile cancer, subgroups of head and neck squamous cell carcinomas (HNSCC), cervical intraepithelial neoplasia (CIN), non-melanoma skin cancer, genital condyloma and recurrent respiratory papillomatosis (RRP).

As used herein, “sample” refers to any sample that contains no LC, contains normal LC, or contains LC exposed to HPV. Such samples include, cell, tissue, blood, mucus, saliva, sweat, vaginal discharge, urine, or fecus.

A “subject” of diagnosis or treatment is a cell, tissue, or a mammal, including a human. Non-human animals subject to diagnosis or treatment include, for example, murine, such as rats, mice, canine, such as dogs, leporids, such as rabbits, livestock, sport animals, and pets. In some embodiments, the “subject” is a HPV-infected patient who may have developed peripheral tolerance towards HPV or a HIV/HPV-infected patients who is slightly more immune compromised.

“Topical administration” refers to delivery of a medication by application to the mucosal membrane or skin. Non-limiting examples of topical administration include any methods described under the definition of “administration” pertaining to delivery of a medication to appropriate area

A penetration or permeation enhancer refers to a chemical composition or mechanical/electrical device that can increase the transdermal drug delivery efficiency. In one aspect, a penetration or permeation enhancer is soluble in the formulation and act to reduce the barrier properties of human skin. The list of potential skin permeation enhancers is long, but can be broken down into three general categories: lipid disrupting agents (LDAs), solubility enhancers, and surfactants. LDAs are typically fatty acid-like molecules proposed to fluidize lipids in the human skin membrane. Solubility enhancers act by increasing the maximum concentration of drug in the formulation, thus creating a larger concentration gradient for diffusion. Surfactants are amphiphilic molecules capable of interacting with the polar and lipid groups in the skin (see e.g. Francoeur et al. (1990) Pharm. Res. 7:621-7; U.S. Pat. No. 5,503,843).

As used herein, “treating” or “treatment” of a disease in a patient refers to (1) preventing the symptoms or disease from occurring in an animal that is predisposed or does not yet display symptoms of the disease; (2) inhibiting the disease or arresting its development; or (3) ameliorating or causing regression of the disease or the symptoms of the disease. As understood in the art, “treatment” is an approach for obtaining beneficial or desired results, including clinical results. For the purposes of this disclosure, beneficial or desired results can include one or more, but are not limited to, preventing, ameliorating, or reducing the likelihood of the development of cancer or preventing, ameliorating, or reducing the pre-cancerous lesions induced by HPV infection, alleviation or amelioration of one or more symptoms, diminishment of extent of a condition (including a disease), stabilized (i.e., not worsening) state of a condition (including disease), delay or slowing of condition (including disease), progression, amelioration or palliation of the condition (including disease), states and remission (whether partial or total), whether detectable or undetectable. Preferred are compounds that are potent and can be administered locally at very low doses, thus minimizing systemic adverse effects.

### B. TLR Agonist

Toll-like receptors (TLRs) are type I transmembrane proteins that allow organisms (including mammals) to detect microbes and initiate an innate immune response (Beutler (2004) Nature 430:257-263). They contain homologous cytoplasmic domains and leucine-rich extracellular domains and typically form homodimers that sense extracellular (or internalized) signals and subsequently initiate a signal transduction cascade via adaptor molecules such as MyD88 (myeloid differentiation factor 88).

TLRs can activate NF-kB and MAP kinases; however, the cytokine/chemokine release profiles derived from TLR activation can be unique to each TLR. Additionally, the signaling pathway that TLRs stimulate may be similar to the pathway induced by the cytokine receptor IL-1R. Once the TLR domain is activated in TLRs and MyD88 is recruited, activation of the IRAK family of serine/threonine kinases results which eventually promotes the degradation of Ik-B and activation of NF-kB (Means et al. (2000) Life Sci. 68:241-258). While it appears that this cascade is designed to allow extracellular stimuli to promote intracellular events, some TLRs may migrate to endosomes where signaling can also be initiated. This process may allow for intimate contact with engulfed microbes and cause innate immune response (Underhill et al. (1999) Nature 401:811-815). This process may also allow host nucleic acids, released by damaged tissues (for example, in inflammatory disease) or apoptosis to trigger a response via endosomal presentation.

Among mammals, there are 11 TLRs that coordinate this rapid response. LC are part of the innate immune system and they express several Toll like receptors (TLRs). TLRs recognize pathogen-associated molecular patterns (PAMPs) and upon engaging their ligands they activate the cell. LC express a variety of TLRs like TLR 1, 2, 3, 5, 6 and 10 (Flacher et al. (2006) J. Immunol. 177:7959-7967).

In some embodiments, the TLR agonist used in the disclosure is one or more of the above recited 11 TLR agonists.

In some embodiments, the TLR agonist used in the disclosure is one or more of the TLR 3, TLR 7, TLR 8, TLR 9, or a combination thereof. In some embodiments, the TLR agonist used in the disclosure is one or more of the TLR 3, TLR 8, TLR 9, or a combination thereof. In some embodiments, the TLR agonist used in the disclosure is one or more of the TLR 8, TLR 9, or a combination thereof. In some embodiments, the TLR agonist used in the disclosure is one or more of the TLR 3, TLR 8, or a combination thereof. In some embodiments, the TLR agonist used in the disclosure is one or more of the TLR 3, TLR 9, or a combination thereof. In some embodiments, the TLR agonist used in the disclosure is TLR 3. In some embodiments, the TLR agonist used in the disclosure is TLR 8. In some embodiments, the TLR agonist used in the disclosure is TLR 9.

In some embodiments, the TLR agonist is a single stranded RNA, double stranded RNA, or a synthetic small molecule.

Examples of TLR 3 agonist include, but are not limited to, polyinosine-polycytidylic acid (poly I:C), a synthetic analog of dsRNA; poly-ICLC; and poly-ICR.

Poly-ICLC drug is a synthetic complex of carboxymethylcellulose, polyinosinic-polycytidylic acid, and poly-L-lysine double-stranded RNA. There are at least four interrelated clinical actions of poly-ICLC, any of which (alone or in combination) might be responsible for its anti-tumor and anti-viral activity. These are 1) its induction of interferons; 2) its broad immune enhancing effect; 3) its activation of specific enzymes, especially oligoadenylate synthetase (OAS) and the p68 protein kinase (PKR); and 4) its broad gene regulatory actions.

Another example of TLR3 agonist is poly-ICR (Poly IC-Poly Arginine), which may have greater biologic effects at much lower concentrations. Poly-ICR is a TLR3 agonist that when combined with a disease-specific antigen can induce both cytotoxic (T-cell) and antibody (B-cell) immune responses against that antigen. Cytotoxic T-cells, also referred to as CD8 T-cells, are required to target and eliminate pathogen-infected or cancerous cells. Antibodies or B-cells, are required to protect against an infection caused by a pathogen. Poly-ICR, therefore, has potential utility in both the therapeutic and prophylactic areas of immunotherapy and vaccine development. This novel and potent immunomodulator works with the immune system to induce dendritic cell maturation, along with a broad range of inflammatory cytokines and chemokines, to facilitate the prevention and treatment of infectious diseases or cancer.

Small molecule examples of TLR 7 agonist include, but are not limited to, CL264 (Adenine analog); Gardiquimod™ (imidazoquinoline compound); Imiquimod (imidazoquinoline compound); and Loxoribine (guanosine analogue).

Examples of TLR 8 agonist include, but are not limited to, single-stranded RNAs and E. coli RNA.

In some embodiments, the TLR agonist activates dual TLR receptors such as, but not limited to, TLR 7/8 agonist. Examples of TLR 7/8 agonist include, but are not limited to, CL075 (3M-002, thiazoloquinoline compound); CL097 (water-soluble R848, imidazoquinoline compound); poly(dT) (thymidine homopolymer phosphorothioate ODN); and R848 (resiquimod, Imidazoquinoline compound).

CL075 (3M002, structure shown below) is a thiazoloquinolone derivative that stimulates TLR8 in human PBMC.

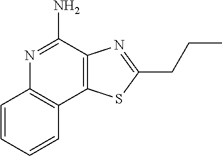

It activates NF-κB and triggers preferentially the production of TNF-α and IL-12. CL075 may also induce the secretion of IFN-α through TLR7 but to a lesser extend. It can induce the activation of NF-κB at 0.4 μM (0.1 μg/ml) in TLR8-transfected HEK293 cells, and ˜10 times more CL075 to activate NF-κB in TLR7-transfected HEK293 cells.

CL097 (structure shown below) is a highly water-soluble derivative of the imidazoquinoline compound R848 (≧20 mg/ml).

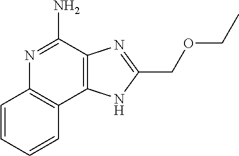

Similarly to R848, CL097 is a TLR7 and TLR8 ligand. It can induce the activation of NF-κB at 0.4 μM (0.1 μg/ml) in TLR7-transfected HEK293 cells and at 4 μM (1 μg/ml) in TLR8-transfected HEK293 cells.

Poly(dT), a thymidine homopolymer phosphorothioate ODN, is a modulator of human TLR7 and TLR8. In combination with an imidazoquinoline, such as R848 and CL075, it increases TLR8-mediated signaling but abolishes TLR7-mediated signaling. A co-incubation of poly(dT) and an imidazoquinoline can induce NF-κB activation in HEK293 cells transfected with murine TLR8- and primary TLR8-expressing mouse cells.

R848 (structure shown below) is an imidazoquinoline compound with potent anti-viral activity.

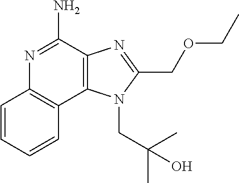

This low molecular weight synthetic molecule activates immune cells via the TLR7/TLR8 MyD88-dependent signaling pathway. R848 has been shown to trigger NF-κB activation in cells expressing murine TLR8 when combined with poly(dT) (Gorden et al. (2006) J. Immunol. 177: 6584-6587).

Toll-like receptor 9 (TLR9) is activated by specific unmethylated CpG-containing sequences in bacterial DNA or synthetic oligonucleotides (ODNs) in the endosomal compartment. These specific sequences called CpG motifs are present at high frequency in bacterial DNA but rare in mammalian DNA. The methylation status is a distinction between bacterial and mammalian DNA. Unmethylated ODNs including a CpG motif can mimic the effects of bacterial DNA, inducing B-cell proliferation and activating cells of the myeloid lineage.

Examples of TLR 9 agonist include, but are not limited to, stimulatory ODNs such as, CpG ODNs, Control ODNs, and Labeled ODNs; and E. coli DNA such as, E. coli DNA of and E. coli ssDNA.

Stimulatory CpG ODNs can be of three types, types A, B and C, which differ in their immune-stimulatory activities. They induce differentially the stimulation of human and murine immune cells in vitro, a species-specificity that is also observed with non-responsive cells such as HEK293 cells transfected with human or mouse TLR9. Type A CpG ODNs are characterized by a phosphodiester central CpG-containing palindromic motif and a phosphorothioate poly-G string. They induce high IFN-a production from plasmacytoid dendritic cells (pDC) but are weak stimulators of TLR9-dependent NF-kappaB signaling. Type B CpG ODNs contain a full phosphorothioate backbone with one or more CpG dinucleotides. They strongly activate B cells but stimulate weakly IFN-α secretion. Type C CpG ODNs combine features of both types A and B. They contain a complete phosphorothioate backbone and a CpG containing palindromic motif. Type C CpG ODNs induce strong IFN-a production from pDC and B cell stimulation.

Control CpG ODNs that do not stimulate TLR9 have been designed for each stimulatory CpG ODN. They feature the same sequence as their stimulatory counterparts but contain GpC dinucleotides in place of CpG dinucleotides.

Stimulatory CpG ODNs are available labeled with FITC at their 3 terminus. FITC-labeled CpG ODNs are useful to study their cellular uptake and localization by confocal laser-scanning microscopy or flow cytometry.

Unlike mammalian DNA, bacterial DNA is rich in unmethylated CpG motifs and thus activates TLR9. E. coli DNA can be of two types, double-stranded DNA and single-stranded DNA complexed with a cationic lipid. E. coli DNA ef is an ultrapure, endotoxin-free (ef) preparation of E. coli K12 double-stranded DNA devoid of TLR2 and TLR4 activities. E. coli ssDNA is an ultrapure, endotoxin-free preparation of bacterial single-stranded DNA (ssDNA). In E. coli ssDNA, TLR9 binds directly and sequence-specifically to single-stranded unmethylated CpG-DNA. E. coli ssDNA is complexed with the cationic lipid LyoVec™ to allow a better internalization of the immunostimulatory DNA to the acidic compartment where TLR9 is expressed. E. coli DNA ef is an ultrapure, endotoxin-free (ef) preparation of E. coli K12 double-stranded DNA devoid of TLR2 and TLR4 activities.

E. coli DNA ef and E. coli ssDNA are provided lyophilized and shipped at room temperature. Store at −20° C. Lyophilized E. coli DNAs are stable 6 months at −20° C.

Other TLR agonists described in US Application Publication Number 2008/0306050, filed Aug. 17, 2006 and US Application Publication Number 2008/0234251, filed Aug. 17, 2006, are incorporated herein by reference in their entirety.

### C. Methods

In one aspect of the disclosure, there is provided a method of activating a Langerhans cell (LC) exposed to a human papillomavirus (HPV), comprising, or alternatively consisting essentially of, or yet further consisting of: administering to a subject an effective amount of a toll-like receptor (TLR) agonist, thereby activating the LC exposed to the HPV. In one aspect, the LC is activated to induce a HPV-specific immune response.

In one aspect of the disclosure, there is provided a method of treating a disease in a subject wherein said disease is responsive to an activation of a Langerhans cell (LC) exposed to a human papillomavirus (HPV), comprising, or alternatively consisting essentially of, or yet further consisting of: administering to a subject an effective amount of a toll-like receptor (TLR) agonist, thereby treating the disease in the subject. The disease includes, but is not limited to, HPV infection or the precancerous lesions induced by the HPV infection.

In one aspect of the disclosure, there is provided a method to reverse human papillomavirus (HPV) immune escape in a subject, comprising, or alternatively consisting essentially of, or yet further consisting of, administering to a subject an effective amount of a toll-like receptor (TLR) agonist, thereby reversing the HPV immune escape in the subject.

HPV infects the epidermal layer of the mucosa where Langerhans cells (LC) are the primary APC. Since LC may be the only APC that HPV can come into contact with during an infection, they are responsible for initiating a cell-mediated immune response against HPV. However, Applicants have previously demonstrated that human LC do not initiate a specific anti-HPV16 CD8⁺ T cell response after exposure to chimeric HPV16L1L2-E7 virus-like particles (HPV16 cVLP) (Fausch et al. (2002) J. Immunol. 169: 3242-3249 and Fausch et al. (2003) Cancer Res. 63. 3478-3482).

Additionally, LC exposed to HPV16L1L2 virus-like particles (HPV16 VLP) may have a tolerizing phenotype, cross-presenting HPV peptides on MHC molecules in the absence of surface markers important for T cell costimulation and migration, including CD80, CD86, and CCR7, and without secretion of proinflammatory cytokines. The molecular mechanism mediating this immune escape process is the activation of PI3K in LC (Fausch (2002) supra; Fausch (2003) supra; and Fausch et al. (2005) J. Immunol. 174: 7172-7178). As a result, HPV can evade the immune system, leading to the delay or absence of viral clearance.

Example 1 herein demonstrates that LC express TLR7 and TLR8. Thus, a potential therapy of HPV16-induced lesions can be to activate HPV16-infected LC using synthetic imidazoquinolines (imiquimod, resiquimod, 3M-002, and 3M-031). Imidazoquinolines are TLR7 and/or TLR8 agonists and therefore are potent innate immune modulators (Table I and Schon and Schon (2008) Oncogene 27: 190-199).

TABLE 1
Synthetic imidazoquinolines and the respective
receptor(s) they bind and act through

| Imidzoquinoline | Agonist receptor(s) |
|---|---|
| 3M-006 | Inactive analog (TLR7/8) |
| 3M-002 | TLR8 |
| Imiquimod | TLR7 |
| Reiquimod | TLR8/7 |
| 3M-031 | TLR7/8 |

TLR7 and TLR8 are localized to endosomal membranes and naturally recognize ssRNA (Schon and Schon supra and Barton (2007) Semin. Immunol. 19: 33-40). Once TLR7 and/or TLR8 are engaged, NF-κB and other transcription factors are activated, leading to the transcription of immune response-related genes, including cytokine, chemokine, costimulatory marker, and adhesion molecule genes (Schon and Schon supra; Gorden et al. (2005) J. Immunol. 174:1259-1268; and Medzhitov et al. (1997) Nature 388: 394-397). Moreover, imidazoquinolines demonstrate antiviral and antitumor activity through cytokines and chemokines, such as TNF-α, IL-6, IL-8, IL-12, and IFN-inducible protein 10 (IP 10), produced by dendritic cells (DC) and macrophages (Schon and Schon supra; Gibson et al. (2002) Cell. Immunol. 218: 74-86; Sauder (2003) Br. J. Dermatol. 149: 5-8. 27; and Wagner et al. (1997) Cytokine 9: 837-845; Weeks et al. (1994) J. Interferon Cytokine Res. 14: 81-85; and Sidky et al. (1992) Cancer Res. 52: 3528-3533).

Without being limited by any theory, Applicants demonstrate that synthetic imidazoquinolines would activate LC previously exposed to HPV16, leading to the induction of an HPV16-specific immune response. The results indicate that select imidazoquinolines, TLR8 dominant agonists, are promising therapeutic drugs that could be used as a treatment for HPV infections and HPV-induced cervical lesions by inducing an anti-HPV-specific cell-mediated immune response via the activation of HPV-infected LC.

Accordingly, in another aspect, there is provided a method of treating an acute or persistent human papillomavirus (HPV) infection in a subject, comprising, or alternatively consisting essentially of, or yet further consisting of: administering to a subject an effective amount of a toll-like receptor (TLR) agonist, thereby treating the acute or persistent HPV infection in the subject.

Acute HPV infection may be reflected in a minor abnormalities in cervical cytology, whereas persistent HPV infection may be a marker for risk of progression to low grade or high grade cervical lesions.

Accordingly, in another aspect, there is provided a method of treating pre-cancerous lesions induced by HPV infection in a subject, comprising, or alternatively consisting essentially of, or yet further consisting of: administering to a subject an effective amount of a toll-like receptor (TLR) agonist, thereby treating the pre-cancerous lesions induced by HPV infection in the subject. In some embodiments, the pre-cancerous lesions induced by HPV infection are at the mucosal surface. In some embodiments, the pre-cancerous lesions induced by HPV infection are at the mucosal surface of the human genital system.

HPV-related cancers are prevalent in HIV-infected individuals, there is a need to develop therapeutic strategies to reduce the risk and prevent the development of HPV-associated malignancies. Since the current HPV preventive vaccines have no therapeutic effect, alternative solutions are needed for this increasing population of HIV individuals co-infected with multiple and persistent HPV types. In yet another aspect, there is provided a method of treating pre-cancerous lesions induced by HPV infection in an HIV infected subject, comprising, consisting of, or consisting essentially of: administering to a subject an effective amount of a toll-like receptor (TLR) agonist, thereby treating the pre-cancerous lesions induced by HPV infection in the HIV infected subject. TLR agonists of the disclosure can reverse HPV immune escape, thus facilitating clearance of persistent HPV infection in both the general population and in HIV-infected individuals.

In another aspect, there is provided an in vitro method for activating a Langerhans cell (LC) exposed to a human papillomavirus (HPV) to induce a HPV-specific immune response, by contacting the LC exposed to HPV with a composition comprising an effective amount of a toll-like receptor (TLR) agonist, and assaying the induced HPV-specific immune response.

In another aspect, there is provided an in vitro method to reverse human papillomavirus (HPV) immune escape, by contacting the LC exposed to HPV with a composition comprising an effective amount of a toll-like receptor (TLR) agonist, and assaying the reversal of the human papillomavirus (HPV) immune escape. In some embodiments, the reversal of the HPV immune escape can be analyzed by analyzing HPV-specific immune response by LC exposed to HPV.

In yet another aspect, there is provided a method for screening of toll-like receptor (TLR) agonist for the treatment of an acute or persistent human papillomavirus (HPV) infection or a pre-cancerous lesion induced by HPV infection, comprising, or alternatively consisting essentially of, or yet further consisting of:

(i) administering a toll-like receptor (TLR) agonist to a test sample containing a Langerhans cell (LC) exposed to a human papillomavirus (HPV) to induce a HPV-specific immune response;

(ii) determining a level of HPV-specific immune response or determining a presence or absence of HPV DNA sequences and/or viral replication; and

thereby screening for TLR agonist for the treatment of an acute or persistent human papillomavirus (HPV) infection or a pre-cancerous lesion induced by HPV infection.

The level of the HPV-specific immune response can be determined by methods well known in the art. Examples of such methods include, but are not limited to, secretion of cytokines, such as interferon gamma and IL-2, by CD8⁺ T lymphocytes upon stimulation with HPV antigens, enumeration of cytokine-secreting T lymphocytes, cellular proliferation of CD4⁺ T lymphocytes specific for HPV antigens, apoptosis induction in cells expressing HPV proteins after co-culture with HPV-specific T lymphocytes, and enumeration of HPV-specific T lymphocytes in samples through the use of recombinant tetramer or pentamer MHC:peptide technology. The presence of HPV infection is cells or tissues can be determined by common molecular biology techniques. Examples of such methods include, but are not limited to, sequence specific polymerase chain reaction (PCR) techniques, oligonucleotide hybridization, in situ hybridization, and immunohistochemistry techniques.

The presence or absence of HPV DNA sequences can be determined by methods well known in the art, e.g. PCR (polymerase chain reaction) technique.

In some embodiments, the method of screening further comprises a control sample where one or more of a toll-like receptor (TLR) agonist is not added to the control sample. In some embodiments, the method of screening further comprises a control sample where the LC is normal or is not exposed to HPV. In some embodiments of the method of screening, the determining step comprises comparing the level of HPV-specific immune response in the sample with the level of HPV-specific immune response in the control sample.

In yet another aspect, there is provided a method of treating human papillomavirus (HPV) infection or a pre-cancerous lesion induced by HPV infection in a subject, comprising, or alternatively consisting essentially of, or yet further consisting of: administering to a subject an effective amount of a toll-like receptor (TLR) agonist in combination with another therapy selected from the group consisting of inflammatory agent, analgesic, or anti-human immunodeficiency virus (HIV) agent, thereby treating the human papillomavirus (HPV) infection or a pre-cancerous lesion induced by HPV infection in the subject. In one aspect, the HPV infection is an acute or persistent HPV infection.

An inflammatory agent can be any agent that induces inflammation. Inflammation can be caused by a physical ablation of tissue or by injury to a tissue. Inflammation involves infiltration of white blood cells into tissue and phagocytosis by white blood cells and can be accompanied by accumulation of pus and an increase in the local temperature.

A local inflammatory response can be accompanied by systemic changes: fever, malaise, an increase in circulating leukocytes (leukocytosis), and increases in specific circulating proteins called acute-phase reactants. The process of inflammation, both vascular and cellular, can be due to an array of molecules produced locally. These mediators include histamine, leukotrienes, prostaglandins, complement components, kinins, antibodies, and interleukins.

Examples of anti-HIV agents include, but are not limited to, nucleoside and nucleotide reverse transcriptase (RT) inhibitors; non-nucleoside reverse transcriptase inhibitors; protease inhibitors (PIs); viral absorption inhibitors; and viral coreceptor agonists. Examples of nucleoside and nucleotide reverse transcriptase (RT) inhibitors include, but are not limited to, nucleoside analog such as zidovudine; and nucleotide analog. Examples of non-nucleoside reverse transcriptase inhibitors include, but are not limited to, non-nucleoside analog such as, but not limited to, nevirapine, delavirdine, and efavirenz. Examples of PIs include, but are not limited to, HIV protease and ABT-378 or lopinavir. Examples of viral absorption inhibitors include, but are not limited to, Cosalane. Examples of viral coreceptor agonists include, but are not limited to, bicyclams.

Examples of analgesics include, but are not limited to, paracetamol (para-acetylaminophenol, also known in the US as acetaminophen); a non-steroidal anti-inflammatory drugs (NSAIDs) such as, but not limited to, the salicylates; COX-2 inhibitors, such as, but not limited to, rofecoxib and celecoxib; opiates and morphinomimetics such as, but not limited to, morphine, the archetypal opioid, and various other substances (e.g. codeine, oxycodone, hydrocodone, diamorphine, pethidine); and synthetic drugs with narcotic properties such as tramadol, and various others.

### D. Pharmaceutical Formulations and Kits

In one aspect, there is provided a pharmaceutical formulation for a treatment of an acute or persistent human papillomavirus (HPV) infection or a pre-cancerous lesion induced by HPV infection in a subject, using an effective amount of a toll-like receptor (TLR) agonist and a pharmaceutically acceptable carrier. The TLR agonists of the present disclosure can be formulated in the pharmaceutical compositions per se, or in the form of a hydrate, solvate, N-oxide, or pharmaceutically acceptable salt, as described herein. Typically, such salts are more soluble in aqueous solutions than the corresponding free acids and bases, but salts having lower solubility than the corresponding free acids and bases may also be formed. The present disclosure includes within its scope solvates of the compounds and salts thereof, for example, hydrates. The compounds may have one or more asymmetric centers and may accordingly exist both as enantiomers and as diastereoisomers. It is to be understood that all such isomers and mixtures thereof are encompassed within the scope of the present disclosure.

In a further aspect, the composition further comprises, or alternatively consists essentially of, or yet further consists of, one or more of an inflammatory agent, an analgesic, or an anti-human immunodeficiency virus (HIV) agent. The anti-HIV agent, in some aspects, is selected from the group of nucleoside and nucleotide reverse transcriptase (RT) inhibitors; non-nucleoside reverse transcriptase inhibitors; protease inhibitors (PIs); viral absorption inhibitors; or viral coreceptor agonists. In some aspects, the analgesic is selected from the group of paracetamol, non-steroidal anti-inflammatory drug, COX-2 inhibitor, opiate or morphinomimetic.

In one embodiment, this disclosure provides a pharmaceutical formulation comprising a TLR agonist alone or in combination with one or more of an inflammatory agent, an analgesic, or an anti-human immunodeficiency virus (HIV) agent. The anti-HIV agent, in some aspects, is selected from the group of nucleoside and nucleotide reverse transcriptase (RT) inhibitors; non-nucleoside reverse transcriptase inhibitors; protease inhibitors (PIs); viral absorption inhibitors; or viral coreceptor agonists. In some aspects, the analgesic is selected from the group of paracetamol, non-steroidal anti-inflammatory drug, COX-2 inhibitor, opiate or morphinomimetic and at least one pharmaceutically acceptable excipient, diluent, preservative, stabilizer, or mixture thereof.

In one embodiment, the methods can be practiced as a therapeutic approach towards the treatment of the conditions described herein. Thus, in a specific embodiment, the compositions comprising the TLR agonist can be used to treat the conditions described herein in animal subjects, including humans. The methods generally comprise administering to the subject an amount of a TLR agonist, effective to treat the condition.

In some embodiments, the subject is a non-human mammal, including, but not limited to, bovine, horse, feline, canine, rodent, or primate. In another embodiment, the subject is a human.

The compounds and compositions of the disclosure can be provided in a variety of formulations and dosages. It is to be understood that reference to the TLR agonist, or “active” in discussions of formulations is also intended to include, where appropriate as known to those of skill in the art, formulation of the TLR agonist, alone or in combination with one or more of an inflammatory agent, an analgesic, or an anti-human immunodeficiency virus (HIV) agent. The anti-HIV agent, in some aspects, is selected from the group of nucleoside and nucleotide reverse transcriptase (RT) inhibitors; non-nucleoside reverse transcriptase inhibitors; protease inhibitors (PIs); viral absorption inhibitors; or viral coreceptor agonists. In some aspects, the analgesic is selected from the group of paracetamol, non-steroidal anti-inflammatory drug, COX-2 inhibitor, opiate or morphinomimetic.

In one embodiment, the TLR agonists are provided as non-toxic pharmaceutically acceptable salts. Suitable pharmaceutically acceptable salts of the TLR agonist include acid addition salts such as those formed with hydrochloric acid, fumaric acid, p-toluenesulphonic acid, maleic acid, succinic acid, acetic acid, citric acid, tartaric acid, carbonic acid, or phosphoric acid. Salts of amine groups may also comprise quaternary ammonium salts in which the amino nitrogen atom carries a suitable organic group such as an alkyl, alkenyl, alkynyl, or substituted alkyl moiety. Furthermore, where the compounds of the disclosure carry an acidic moiety, suitable pharmaceutically acceptable salts thereof may include metal salts such as alkali metal salts, e.g., sodium or potassium salts; and alkaline earth metal salts, e.g., calcium or magnesium salts.

The pharmaceutically acceptable salts of the TLR agonist can be formed by conventional means, such as by reacting the free base form of the product with one or more equivalents of the appropriate acid in a solvent or medium in which the salt is insoluble or in a solvent such as water which is removed in vacuo, by freeze drying, or by exchanging the anions of an existing salt for another anion on a suitable ion exchange resin.

Pharmaceutical compositions comprising the TLR agonist described herein can be manufactured by means of conventional mixing, dissolving, granulating, dragee-making levigating, emulsifying, encapsulating, entrapping, or lyophilization processes. The compositions can be formulated in conventional manner using one or more physiologically acceptable carriers, diluents, excipients, or auxiliaries which facilitate processing of the active compounds into preparations which can be used pharmaceutically.

The TLR agonist of the disclosure can be administered by oral, parenteral (e.g., intramuscular, intraperitoneal, intravenous, ICV, intracisternal injection or infusion, subcutaneous injection, or implant), by inhalation spray nasal, vaginal, rectal, sublingual, urethral (e.g., urethral suppository) or topical routes of administration (e.g., gel, ointment, cream, aerosol, etc.) and can be formulated, alone or together, in suitable dosage unit formulations containing conventional non-toxic pharmaceutically acceptable carriers, adjuvants, excipients, and vehicles appropriate for each route of administration.

The pharmaceutical compositions for the administration of the TLR agonist can be conveniently presented in dosage unit form and can be prepared by any of the methods well known in the art of pharmacy. The pharmaceutical compositions can be, for example, prepared by uniformly and intimately bringing the active ingredient into association with a liquid carrier, a finely divided solid carrier or both, and then, if necessary, shaping the product into the desired formulation. In the pharmaceutical composition the active is included in an amount sufficient to produce the desired therapeutic effect. For example, pharmaceutical compositions of the disclosure may take a form suitable for virtually any mode of administration, including, for example, topical, ocular, oral, buccal, systemic, nasal, injection, transdermal, rectal, and vaginal, or a form suitable for administration by inhalation or insufflation.

For topical administration, the compound(s) or prodrug(s) can be formulated as solutions, gels, ointments, creams, suspensions, etc., as is well-known in the art.

Systemic formulations include those designed for administration by injection (e.g., subcutaneous, intravenous, intramuscular, intrathecal, or intraperitoneal injection) as well as those designed for transdermal, transmucosal, oral, or pulmonary administration.

Useful injectable preparations include sterile suspensions, solutions, or emulsions of the active compound(s) in aqueous or oily vehicles. The compositions may also contain formulating agents, such as suspending, stabilizing, and/or dispersing agents. The formulations for injection can be presented in unit dosage form, e.g., in ampules or in multidose containers, and may contain added preservatives.

Alternatively, the injectable formulation can be provided in powder form for reconstitution with a suitable vehicle, including but not limited to sterile pyrogen free water, buffer, and dextrose solution, before use. To this end, the active compound(s) can be dried by any art-known technique, such as lyophilization, and reconstituted prior to use.

For transmucosal administration, penetrants appropriate to the barrier to be permeated are used in the formulation. Such penetrants are known in the art.

For oral administration, the pharmaceutical compositions may take the form of, for example, lozenges, tablets, or capsules prepared by conventional means with pharmaceutically acceptable excipients such as binding agents (e.g., pregelatinised maize starch, polyvinylpyrrolidone, or hydroxypropyl methylcellulose); fillers (e.g., lactose, microcrystalline cellulose, or calcium hydrogen phosphate); lubricants (e.g., magnesium stearate, talc, or silica); disintegrants (e.g., potato starch or sodium starch glycolate); or wetting agents (e.g., sodium lauryl sulfate). The tablets can be coated by methods well known in the art with, for example, sugars, films, or enteric coatings. Additionally, the pharmaceutical compositions containing the TLR agonist as active ingredient or prodrug thereof in a form suitable for oral use may also include, for example, troches, lozenges, aqueous, or oily suspensions, dispersible powders or granules, emulsions, hard or soft capsules, or syrups or elixirs.

Compositions intended for oral use can be prepared according to any method known to the art for the manufacture of pharmaceutical compositions, and such compositions may contain one or more agents selected from the group consisting of sweetening agents, flavoring agents, coloring agents, and preserving agents in order to provide pharmaceutically elegant and palatable preparations. Tablets contain the active ingredient (including drug and/or prodrug) in admixture with non-toxic pharmaceutically acceptable excipients which are suitable for the manufacture of tablets. These excipients can be for example, inert diluents, such as calcium carbonate, sodium carbonate, lactose, calcium phosphate or sodium phosphate; granulating and disintegrating agents (e.g., corn starch or alginic acid); binding agents (e.g. starch, gelatin, or acacia); and lubricating agents (e.g., magnesium stearate, stearic acid, or talc). The tablets can be left uncoated or they can be coated by known techniques to delay disintegration and absorption in the gastrointestinal tract and thereby provide a sustained action over a longer period. For example, a time delay material such as glyceryl monostearate or glyceryl distearate can be employed. They may also be coated by the techniques described in the U.S. Pat. Nos. 4,256,108; 4,166,452; and 4,265,874 to form osmotic therapeutic tablets for control release. The pharmaceutical compositions of the disclosure may also be in the form of oil-in-water emulsions.

Liquid preparations for oral administration may take the form of, for example, elixirs, solutions, syrups, or suspensions, or they can be presented as a dry product for constitution with water or other suitable vehicle before use. Such liquid preparations can be prepared by conventional means with pharmaceutically acceptable additives such as suspending agents (e.g., sorbitol syrup, cellulose derivatives, or hydrogenated edible fats); emulsifying agents (e.g., lecithin, or acacia); non-aqueous vehicles (e.g., almond oil, oily esters, ethyl alcohol, Cremophore™, or fractionated vegetable oils); and preservatives (e.g., methyl or propyl-p-hydroxybenzoates or sorbic acid). The preparations may also contain buffer salts, preservatives, flavoring, coloring, and sweetening agents as appropriate.

Preparations for oral administration can be suitably formulated to give controlled release or sustained release of the active compound, as is well known. The sustained release formulations of this disclosure are preferably in the form of a compressed tablet comprising an intimate mixture of compound of the disclosure and a partially neutralized pH-dependent binder that controls the rate of compound dissolution in aqueous media across the range of pH in the stomach (typically approximately 2) and in the intestine (typically approximately about 5.5).

To provide for a sustained release of compounds of the disclosure, one or more pH-dependent binders can be chosen to control the dissolution profile of the sustained release formulation so that the formulation releases compound slowly and continuously as the formulation is passed through the stomach and gastrointestinal tract. Accordingly, the pH-dependent binders suitable for use in this disclosure are those which inhibit rapid release of drug from a tablet during its residence in the stomach (where the pH is-below about 4.5), and which promotes the release of a therapeutic amount of the compound of the disclosure from the dosage form in the lower gastrointestinal tract (where the pH is generally greater than about 4.5). Many materials known in the pharmaceutical art as “enteric” binders and coating agents have a desired pH dissolution properties. The examples include phthalic acid derivatives such as the phthalic acid derivatives of vinyl polymers and copolymers, hydroxyalkylcelluloses, alkylcelluloses, cellulose acetates, hydroxyalkylcellulose acetates, cellulose ethers, alkylcellulose acetates, and the partial esters thereof, and polymers and copolymers of lower alkyl acrylic acids and lower alkyl acrylates, and the partial esters thereof. One or more pH-dependent binders present in the sustained release formulation of the disclosure are in an amount ranging from about 1 to about 20 wt %, more preferably from about 5 to about 12 wt % and most preferably about 10 wt %.

One or more pH-independent binders may be in used in oral sustained release formulation of the disclosure. The pH-independent binders can be present in the formulation of this disclosure in an amount ranging from about 1 to about 10 wt %, and preferably in amount ranging from about 1 to about 3 wt % and most preferably about 2 wt %.

The sustained release formulation of the disclosure may also contain pharmaceutical excipients intimately admixed with the compound and the pH-dependent binder. Pharmaceutically acceptable excipients may include, for example, pH-independent binders or film-forming agents such as hydroxypropyl methylcellulose, hydroxypropyl cellulose, methylcellulose, polyvinylpyrrolidone, neutral poly(meth)acrylate esters, starch, gelatin, sugars, carboxymethylcellulose, and the like. Other useful pharmaceutical excipients include diluents such as lactose, mannitol, dry starch, microcrystalline cellulose and the like; surface active agents such as polyoxyethylene sorbitan esters, sorbitan esters and the like; and coloring agents and flavoring agents. Lubricants (such as talc and magnesium stearate) and other tableting aids can also be optionally present.

The sustained release formulations of this disclosure have a TLR agonist of this disclosure in the range of about 50% by weight to about 95% or more by weight, and preferably between about 70% to about 90% by weight; a pH-dependent binder content of between 5% and 40%, preferably between 5% and 25%, and more preferably between 5% and 15%; with the remainder of the dosage form comprising pH-independent binders, fillers, and other optional excipients.

In some embodiments, the topical or oral formulations of TLR agonists are within the range of about 1-10% wt/vol. In some embodiments, the non-topical formulations of TLR agonists are within the range of about 500-1500 microgram per injection.

For buccal administration, the compositions may take the form of tablets or lozenges formulated in the conventional manner.

For rectal and vaginal routes of administration, the active compound(s) can be formulated as solutions (for retention enemas), suppositories, or ointments containing conventional suppository bases such as cocoa butter or other glycerides.

For nasal administration or administration by inhalation or insufflation, the active compound(s) or prodrug(s) can be conveniently delivered in the form of an aerosol spray from pressurized packs or a nebulizer with the use of a suitable propellant (e.g., dichlorodifluoromethane, trichlorofluoromethane, dichlorotetrafluoroethane, fluorocarbons, carbon dioxide, or other suitable gas). In the case of a pressurized aerosol, the dosage unit can be determined by providing a valve to deliver a metered amount. Capsules and cartridges for use in an inhaler or insufflator (for example, capsules and cartridges comprised of gelatin) can be formulated containing a powder mix of the compound and a suitable powder base such as lactose or starch.

The pharmaceutical compositions can be in the form of a sterile injectable aqueous or oleaginous suspension. This suspension can be formulated according to the known art using those suitable dispersing or wetting agents and suspending agents which have been mentioned above. The sterile injectable preparation may also be a sterile injectable solution or suspension in a non-toxic parenterally-acceptable diluent or solvent. Among the acceptable vehicles and solvents that can be employed are water, Ringer's solution, and isotonic sodium chloride solution. The compounds may also be administered in the form of suppositories for rectal or urethral administration of the drug.

For topical use, creams, ointments, jellies, gels, solutions, suspensions, etc., containing the compounds of the disclosure, can be employed. In some embodiments, the TLR agonist can be formulated for topical administration. In some embodiments, the TLR agonist can be formulated for topical administration with polyethylene glycol (PEG). These formulations may optionally comprise additional pharmaceutically acceptable ingredients such as diluents, stabilizers, and/or adjuvants.

In one embodiment, the TLR agonist of the present disclosure can be administered topically, such as through a skin patch, a semi-solid, or a liquid formulation, for example a gel, a (micro-) emulsion, an ointment, a solution, a (nano/micro)-suspension, or a foam. The penetration of the drug into the skin and underlying tissues can be regulated, for example, using penetration enhancers; the appropriate choice and combination of lipophilic, hydrophilic, and amphiphilic excipients, including water, organic solvents, waxes, oils, synthetic and natural polymers, surfactants, emulsifiers; by pH adjustment; and use of complexing agents. Other techniques, such as iontophoresis, may be used to regulate skin penetration of a compound of the disclosure. Transdermal or topical administration would be preferred, for example, in situations in which local delivery with minimal systemic exposure is desired.

Pharmaceutical formulations adapted for topical administration may be provided as ointments, creams, suspensions, lotions, powders, solutions, pastes, gels, sprays, aerosols or oils. For topical administration to the skin, mouth, eye or other external tissues a topical ointment or cream is preferably used. When formulated in an ointment, the active ingredient(s) may be employed with either a paraffinic or a water-miscible ointment base. Alternatively, the active ingredient(s) may be formulated in a cream with an oil-in-water base or a water-in-oil base. Pharmaceutical formulations adapted for topical administration to the eye include eye drops. Here the active ingredient(s) can be dissolved or suspended in a suitable carrier, e.g. in an aqueous solvent. Pharmaceutical formulations adapted for topical administration in the mouth include lozenges, pastilles and mouthwashes.

Included among the devices which can be used to administer compounds of the disclosure, are those well-known in the art, such as metered dose inhalers, liquid nebulizers, dry powder inhalers, sprayers, thermal vaporizers, and the like. Other suitable technology for administration of particular TLR agonist of the disclosure, includes electrohydrodynamic aerosolizers. As those skilled in the art will recognize, the formulation of TLR agonist, the quantity of the formulation delivered, and the duration of administration of a single dose depend on the type of inhalation device employed as well as other factors. For some aerosol delivery systems, such as nebulizers, the frequency of administration and length of time for which the system is activated will depend mainly on the concentration of compounds in the aerosol. For example, shorter periods of administration can be used at higher concentrations of compounds in the nebulizer solution. Devices such as metered dose inhalers can produce higher aerosol concentrations and can be operated for shorter periods to deliver the desired amount of TLR agonist in some embodiments. Devices such as dry powder inhalers deliver active agent until a given charge of agent is expelled from the device. In this type of inhaler, the amount of TLR agonist in a given quantity of the powder determines the dose delivered in a single administration.

Formulations of the TLR agonist of the disclosure for administration from a dry powder inhaler may typically include a finely divided dry powder containing compounds, but the powder can also include a bulking agent, buffer, carrier, excipient, another additive, or the like. Additives can be included in a dry powder formulation of compounds of the disclosure, for example, to dilute the powder as required for delivery from the particular powder inhaler, to facilitate processing of the formulation, to provide advantageous powder properties to the formulation, to facilitate dispersion of the powder from the inhalation device, to stabilize to the formulation (e.g., antioxidants or buffers), to provide taste to the formulation, or the like. Typical additives include mono-, di-, and polysaccharides; sugar alcohols and other polyols, such as, for example, lactose, glucose, raffinose, melezitose, lactitol, maltitol, trehalose, sucrose, mannitol, starch, or combinations thereof; surfactants, such as sorbitols, diphosphatidyl choline, or lecithin; and the like.

For prolonged delivery, the TLR agonist can be formulated as a depot preparation for administration by implantation or intramuscular injection. The active ingredient can be formulated with suitable polymeric or hydrophobic materials (e.g., as an emulsion in an acceptable oil) or ion exchange resins, or as sparingly soluble derivatives (e.g., as a sparingly soluble salt). Alternatively, transdermal delivery systems manufactured as an adhesive disc or patch which slowly releases the active compound(s) for percutaneous absorption can be used. To this end, permeation enhancers can be used to facilitate transdermal penetration of the active compound(s). Suitable transdermal patches are described in, for example, U.S. Pat. No. 5,407,713; U.S. Pat. No. 5,352,456; U.S. Pat. No. 5,332,213; U.S. Pat. No. 5,336,168; U.S. Pat. No. 5,290,561; U.S. Pat. No. 5,254,346; U.S. Pat. No. 5,164,189; U.S. Pat. No. 5,163,899; U.S. Pat. No. 5,088,977; U.S. Pat. No. 5,087,240; U.S. Pat. No. 5,008,110; and U.S. Pat. No. 4,921,475.

Alternatively, other pharmaceutical delivery systems can be employed. Liposomes and emulsions are well-known examples of delivery vehicles that can be used to deliver active compound(s) or prodrug(s). Certain organic solvents such as dimethylsulfoxide (DMSO) may also be employed, although usually at the cost of greater toxicity.

The pharmaceutical compositions may, if desired, be presented in a pack or dispenser device which may contain one or more unit dosage forms containing the active compound(s). The pack may, for example, comprise metal or plastic foil, such as a blister pack. The pack or dispenser device can be accompanied by instructions for administration.

The TLR agonist described herein, or compositions thereof, will generally be used in an amount effective to achieve the intended result, for example, in an amount effective to treat or prevent the particular condition being treated. The TLR agonist(s) can be administered therapeutically to achieve therapeutic benefit or prophylactically to achieve prophylactic benefit. By therapeutic benefit is meant eradication or amelioration of the underlying disorder being treated and/or eradication or amelioration of one or more of the symptoms associated with the underlying disorder such that the patient reports an improvement in feeling or condition, notwithstanding that the patient may still be afflicted with the underlying disorder. For example, administration of a TLR agonist to a patient suffering from HPV infection provides therapeutic benefit not only when the HPV infection is eradicated or ameliorated, but also when the patient reports a decrease in the severity or duration of the symptoms associated with the HPV infection. Therapeutic benefit also includes halting or slowing the progression of the disease, regardless of whether improvement is realized.

The amount of TLR agonist administered will depend upon a variety of factors, including, for example, the particular condition being treated, the mode of administration, the severity of the condition being treated, the age and weight of the patient, the bioavailability of the particular active compound. Determination of an effective dosage is well within the capabilities of those skilled in the art. As known by those of skill in the art, the preferred dosage of compounds of the disclosure will also depend on the age, weight, general health, and severity of the condition of the individual being treated. Dosage may also need to be tailored to the sex of the individual and/or the lung capacity of the individual, where administered by inhalation. Dosage, and frequency of administration of the compounds or prodrugs thereof, will also depend on whether the compounds are formulated for treatment of acute episodes of a condition or for the prophylactic treatment of a disorder. A skilled practitioner will be able to determine the optimal dose for a particular individual.

Effective dosages can be estimated initially from in vitro assays. For example, an initial dosage for use in animals can be formulated to achieve a circulating blood or serum concentration of active compound that is at or above an IC₅₀ of the particular TLR agonist as measured in as in vitro assay. Calculating dosages to achieve such circulating blood or serum concentrations taking into account the bioavailability of the particular compound is well within the capabilities of skilled artisans. For guidance, the reader is referred to Fingl & Woodbury, “General Principles,” GOODMAN AND GILMAN'S THE PHARMACEUTICAL BASIS OF THERAPEUTICS, Chapter 1, pp. 1-46, latest edition, Pergamagon Press, and the references cited therein.

Initial dosages can also be estimated from in vivo data, such as animal models. Animal models useful for testing the efficacy of TLR agonist to treat or prevent the various diseases described above are well-known in the art. Ordinarily skilled artisans can routinely adapt such information to determine dosages suitable for human administration.

Dosage amounts will typically be in the range of from about 0.0001 or 0.001 or 0.01 mg/kg/day to about 100 mg/kg/day, but can be higher or lower, depending upon, among other factors, the activity of the TLR agonist, its bioavailability, the mode of administration, and various factors discussed above. Dosage amount and interval can be adjusted individually to provide plasma levels of the compound(s) which are sufficient to maintain therapeutic or prophylactic effect. For example, the TLR agonist can be administered once per week, several times per week (e.g., every other day), once per day, or multiple times per day, depending upon, among other things, the mode of administration, the specific indication being treated, and the judgment of the prescribing physician. In cases of local administration or selective uptake, such as local topical administration, the effective local concentration of active compound(s) may not be related to plasma concentration. Skilled artisans will be able to optimize effective local dosages without undue experimentation.

Preferably, the TLR agonist(s) will provide therapeutic or prophylactic benefit without causing substantial toxicity. Toxicity of the TLR agonist(s) can be determined using standard pharmaceutical procedures. The dose ratio between toxic and therapeutic (or prophylactic) effect is the therapeutic index. The TLR agonist(s) that exhibit high therapeutic indices are preferred.

The foregoing disclosure pertaining to the dosage requirements for the TLR agonist of the disclosure is pertinent to dosages required for prodrugs, with the realization, apparent to the skilled artisan, that the amount of prodrug(s) administered will also depend upon a variety of factors, including, for example, the bioavailability of the particular prodrug(s) and the conversation rate and efficiency into active drug compound under the selected route of administration. Determination of an effective dosage of prodrug(s) for a particular use and mode of administration is well within the capabilities of those skilled in the art.

Also provided are kits for administration of the TLR agonist of the disclosure, or pharmaceutical formulations comprising the TLR agonist that may include a dosage amount of at least one TLR agonist or a composition comprising at least one TLR agonist, as disclosed herein. In one aspect, there is provided a kit for a treatment of an acute or persistent human papillomavirus (HPV) infection or a pre-cancerous lesion induced by HPV infection in a subject, comprising: an effective amount of a toll-like receptor (TLR) agonist.

Kits may further comprise suitable packaging and/or instructions for use of the TLR agonist. Kits may also comprise a means for the delivery of the at least one TLR agonist or compositions comprising at least one TLR agonist of the disclosure, such as an inhaler, spray dispenser (e.g., nasal spray), syringe for injection, or pressure pack for capsules, tablets, suppositories, or other device as described herein.

Other types of kits provide the TLR agonist and reagents to prepare a composition for administration. The composition can be in a dry or lyophilized form or in a solution, particularly a sterile solution. When the composition is in a dry form, the reagent may comprise a pharmaceutically acceptable diluent for preparing a liquid formulation. The kit may contain a device for administration or for dispensing the compositions, including, but not limited to, syringe, pipette, transdermal patch, or inhalant.

In some embodiments, the pharmaceutically acceptable carrier in the kits is suitable for topical administration of the agent. Additional agents can be co-formulated or delivered concomitantly or sequentially with the above noted agents, as described herein. The formulations can be for immediate or controlled release of the active ingredients.

The kits may include other therapeutic compounds for use in conjunction with the TLR agonist described herein. These compounds can be provided in a separate form or mixed with the TLR agonist of the present disclosure. The kits will include appropriate instructions for preparation and administration of the composition, side effects of the compositions, and any other relevant information. The instructions can be in any suitable format, including, but not limited to, printed matter, videotape, computer readable disk, or optical disc.

In one embodiment, this disclosure provides a kit comprising a TLR agonist selected from the disclosure or a prodrug thereof, packaging, and instructions for use.

In another embodiment, this disclosure provides a kit comprising the pharmaceutical formulation comprising a TLR agonist or a prodrug thereof and at least one pharmaceutically acceptable excipient, diluent, preservative, stabilizer, or mixture thereof, packaging, and instructions for use. In another embodiment, kits for treating an individual who suffers from or is susceptible to the conditions described herein are provided, comprising a container comprising a dosage amount of a TLR agonist of this disclosure or composition, as disclosed herein, and instructions for use. The container can be any of those known in the art and appropriate for storage and delivery of oral, intravenous, topical, rectal, urethral, or inhaled formulations.

The kits will include appropriate instructions for preparation and administration of the composition, side effects of the compositions, and any other relevant information. The instructions can be in any suitable format, including, but not limited to, printed matter, videotape, computer readable disk, or optical disc.

In another aspect of the disclosure, kits for treating an individual who suffers from or is susceptible to the conditions described herein are provided, comprising a container comprising a dosage amount of a composition, as disclosed herein, and instructions for use. The container can be any of those known in the art and appropriate for storage and delivery of oral, intravenous, intravaginally, anal, topical, rectal, urethral, or inhaled formulations.

Kits may also be provided that contain sufficient dosages of the TLR agonists or composition to provide effective treatment for an individual for an extended period, such as a week, 2 weeks, 3, weeks, 4 weeks, 6 weeks, or 8 weeks or more.

The following examples are intended to illustrate the various embodiments of this disclosure.

### EXAMPLES

The disclosure is further understood by reference to the following examples, which are intended to be purely exemplary of the disclosure. The present disclosure is not limited in scope by the exemplified embodiments, which are intended as illustrations of single aspects of the disclosure only. Any methods that are functionally equivalent are within the scope of the disclosure. Various modifications of the disclosure in addition to those described herein will become apparent to those skilled in the art from the foregoing description. Such modifications fall within the scope of the appended claims.

In the examples below as well as throughout the application, the following abbreviations have the following meanings. If not defined, the terms have their generally accepted meanings.

g = gram
h = hour
ng = nanogram
mg = milligram
ml = milliliter
mM = milimolar
ng = nanogram
μg = microgram
μL = microliter
μM = micromolar
U/ml = Units/milliliter

### Example 1

### Materials and Methods

### Antibodies (Abs) and Agonists

The Abs recognizing conformational HPV16 L1 epitopes (H16.V5, H16.E70) or linear HPV16 L1 epitopes (Camvir-1, H16.D9, H16.H5) were gifts from N. Christensen (Penn State, Hershey, Pa.), except Camvir-1, which was purchased from BD Biosciences. Polyclonal serum (DK44214) recognizing HPV16 L2 was a gift from J. Schiller (National Institutes of Health, Bethesda, Md.). The Abs to human CD 197 (CCR7)-PE, CD1a-PE, CD80-FITC, CD86-FITC, HLA-DR, DQ, DP-FITC, HLA-A, B, C-FITC, isotype controls, biotinylated anti-rabbit IgG, streptavidin-PE, and streptavidin-HRP were purchased from BD Biosciences. The Ab to human CD207 (langerin) was purchased from Immunotech and the anti-human E-cadherin Ab was purchased from Millipore. Anti-human TLR7 and antihuman TLR8-PE were purchased from Abcam. Goat anti-rabbit-HRP was purchased from BioSource International. Anti-human IFN-γ and biotinylated anti-human IFN-γ Abs were purchased from Mabtech. TLR7, 8, and 7/8 agonists (3M-006, 3M-002, 3M-005, 3M-007, 3M-031) were gifts from 3M Pharmaceuticals.

### Donor Material

PBL were obtained by leukapheresis from healthy donors. Leukocytes were purified using Lymphocyte Separation Media (Mediatech) by gradient centrifugation, cryopreserved, and stored in liquid nitrogen. HPV serology analysis of all donors showed negative results. All studies using human samples were approved by the University of Southern California's Institutional Review Board and informed consent was obtained from all donors.

### DC and LC Generation

Frozen PBL were thawed and washed once with RPMI 1640 containing 2 mM glutamax (Life Technologies), 10 mM sodium pyruvate (Life Technologies), 10 mM nonessential amino acids (Life Technologies), 100 μg/ml kanamycin (Sigma-Aldrich), and 10% FBS (complete medium; Omega Scientific). For DC, plastic adherent cells were selected by plating 2×10⁸ cells in a 175-cm² tissue culture flask for 2 h at 37° C. Nonadherent cells were washed away and the remaining cells were cultured for 7 days in complete medium containing 1000 U/ml rGM-CSF (Berlex) and 1000 U/ml rIL-4 (BioSource International), of which 100% was replenished on day 3 and 50% was replenished on day 6. For LC, adherent cells were cultured for 7 days in complete medium containing 1000 U/ml rGM-CSF, 1000 U/ml rIL-4, and 10 ng/ml rTGF-β1 (BioSource International), of which 100% was replenished on day 3, 50% of rGM-CSF and rIL-4 was replenished on day 6, and 100% of rTGF-β1 was replenished on days 3 and 6.

### Virus-Like Particles

HPV16L1L2 VLP and HPV16L1L2-E7 cVLP were produced in insect cells and purified by sucrose and cesium chloride ultracentrifugation as previously described (Greenstone et al. (1998) Proc. Natl. Acad. Sci. USA 95: 1800-1805 and Kirnbauer et al. (1993) J. Virol. 67: 6929-6939). Western blot analysis confirmed the presence of L1, L2, and, in the case of chimeric particles, the E7 protein. To test for intact particles, VLP were subjected to an ELISA using Abs that recognize conformationally dependent L1 surface epitopes or linear epitopes, and transmission electron microscopy. An E-toxate kit (Sigma-Aldrich) was used to quantitate endotoxin and levels in the preparations were found to be 1<0.06 endotoxin units/ml. This level as well as baculovirus DNA used in the VLP production procedure do not activate APC (Fausch et al. (2002) J. Immunol. 169: 3242-3249).

### Imidazoquinoline Activation Assay

DC and LC were harvested and washed twice with PBS. DC were left untreated or treated with 30 μM 3M-006, 5 μM 3M-002, 30 μM imiquimod, 30 μM resiquimod, 5 μM 3M-031, or with 10 μg LPS (Escherichia coli 026:B6; Sigma-Aldrich). The cells were incubated for 1 h at 37° C., mixed occasionally, and finally placed at 37° C. for 24 h in complete medium containing 1000 U/ml rGM-CSF. LC were left untreated or exposed to HPV16 VLP at a concentration of 10 μg/10⁶ cells. The cells were incubated for 1 h at 37° C., mixed occasionally, and placed at 37° C. for 24 h in complete medium containing 1000 U/ml rGM-CSF. Next, the cells were left untreated or treated with 30 μM 3M-006, 5 μM 3M-002, 30 μM imiquimod, 30 μM resiquimod, 5 μM 3M-031, or 10 μg of LPS and incubated for an additional 24 h at 37° C. DC and LC were harvested, washed, and analyzed by flow cytometry for the expression and surface markers. Additionally, untreated LC and LC exposed to HPV16 VLP were also analyzed for the expression of TLR7 and TLR8.

### Cytokine and Chemokine Analysis

Supernatants were collected from LC stimulated in the imidazoquinolines activation assay and submitted to the Beckman Center for Immune Monitoring Core at the University of Southern California for cytokine and chemokine analysis. The assays were completed using Human Cytokine LINCOplex Kits (LINCO Research) and the Bio-Plex Suspension Array System (Bio-Rad).

### Migration Assay

Chemokine-directed migration of LC was conducted using 24-well Transwell plates with 5-μm pore size polycarbonate filters (Corning Costar. Briefly, 600 μl of medium was added to the lower chamber containing either 250 ng/ml CCL21 (R&D Systems) or complete medium alone, as a control for spontaneous migration. Applicants added 2×10⁵ untreated LC, LPS-stimulated

LC, HPV16 VLP-exposed LC, or HPV16 VLP-exposed LC treated with each of the imidazoquinolines, using the same concentrations as stated in the imidazoquinoline activation assay, to the upper chambers. The plates were incubated for 3 h at 37° C. Cells that migrated to the lower chamber were counted, and migration was calculated as the ratio of cells that migrated with/without CCL21.

### In Vitro Immunization Assay

In vitro immunizations assays were performed as described previously (Fausch et al. supra and Rudolf et al. (2001) J. Immunol. 166: 5917-5924). Briefly, LC were left untreated or exposed to 10 μg of HPV16 cVLP for 1 h at 37° C. in PBS. Subsequently, the cells were incubated for 4 h in complete medium supplemented with 1000 U/ml rGM-CSF at 37° C. Then cells were treated with or without each of the imidazoquinolines and incubated for 20 h at 37° C. As a control for epitope presentation, imidazoquinoline-treated LC were pulsed with a HLA-A2-restricted HPV16-E7 peptide (aa 86-93) (Ressing et al. (1995) J. Immunol. 154: 5934-5943). LC were irradiated (25 Gy) and mixed with autologous CD8⁺ T cells isolated from PBL by positive selection using a MACS MulitSort CD8⁺ isolation kit (Miltenyi Biotec). Day 7 and 14 re-stimulations were done with LC treated as indicated above. For this, the medium was supplemented with IL-2 at 50 U/ml at 48 and 96 h after re-stimulation. After 28 days, cells were pooled and tested for IFN-γ production by ELISPOT as a measurement of HPV16-E7-specific CD8⁺ T cell responses. Briefly, 96-well multiscreen hemagglutinin plates (Millipore) were coated with 10 μg/ml anti-human IFN-γ in PBS overnight, washed with PBS/0.5% Tween 20, and blocked for 4 h with complete medium at 37° C./5% CO₂. Then, 2.5×10⁵ cells/well were incubated in the presence or absence of HPV16-E7 peptide aa 86-93 for 18 h at 37° C. The wells were washed six times with PBS/0.5% Tween 20 and plates were incubated for 1 h with streptavidin-HRP conjugate diluted in PBS/0.5% BSA solution. Individual spots were counted after staining with 3-amino-9-ethyl-carbazole substrate (Sigma-Aldrich). Spots were counted using the video-imaging KS ELISPOT analysis system (Zeiss).

### Statistical Analysis

All statistical analyses were performed using GraphPad Prism. Statistical analyses of the DC activation assay and ELISPOT assay were conducted using a two-tailed t test, as compared with the negative control. Statistical significance of the LC activation assay, cytokine and chemokine analysis, and migration assay were determined by a one-way ANOVA and Tukey's multiple comparison test as compared with the negative controls.

### Characterization of LC

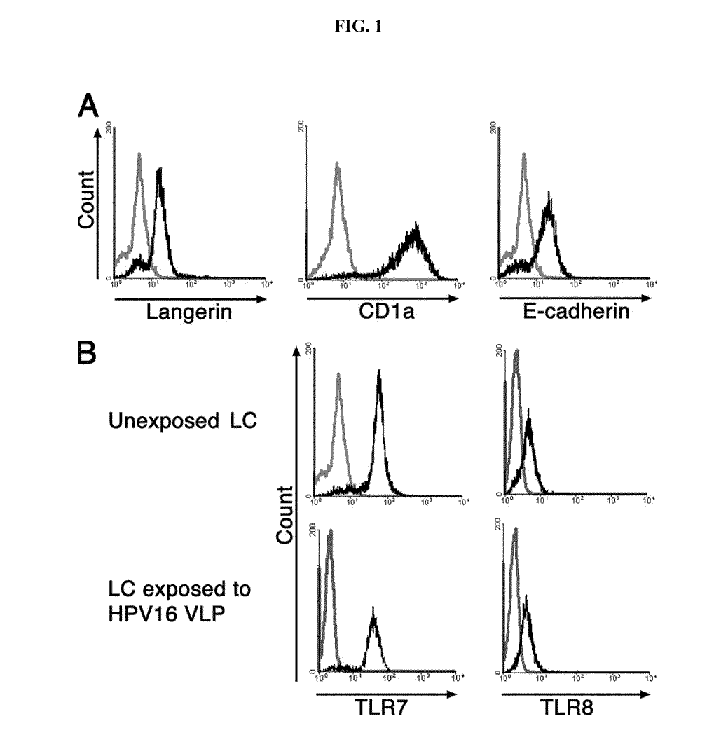

In this study, Applicants examined TLR7 and/or TLR8 agonists as a means to initiate the activation of HPV16-infected LC, thereby inducing an effective cell-mediated immune response against HPV16. To verify the purity of the LC used in this study, Applicants assessed by flow cytometry the presence of surface markers commonly used to identify LC: langerin, CD1a, and E-cadherin. Applicants' results showed that LC generated from human monocytes were a pure population and expressed LC-associated surface markers; therefore, they were phenotypically equivalent to LC found in the epidermis (FIG. 1A). Applicants also analyzed the expression of both TLR7 and TLR8 in immature LC and HPV16 VLP-exposed LC by flow cytometry. The results clearly demonstrate that TLR7 and TLR8 are expressed at similar levels in immature LC and LC exposed to HPV16 VLP (FIG. 1B).

### 3M-002 and Resiquimod Up-Regulate Surface Markers, MHC Class I, MHC Class II, CD80, and CD86 on LC

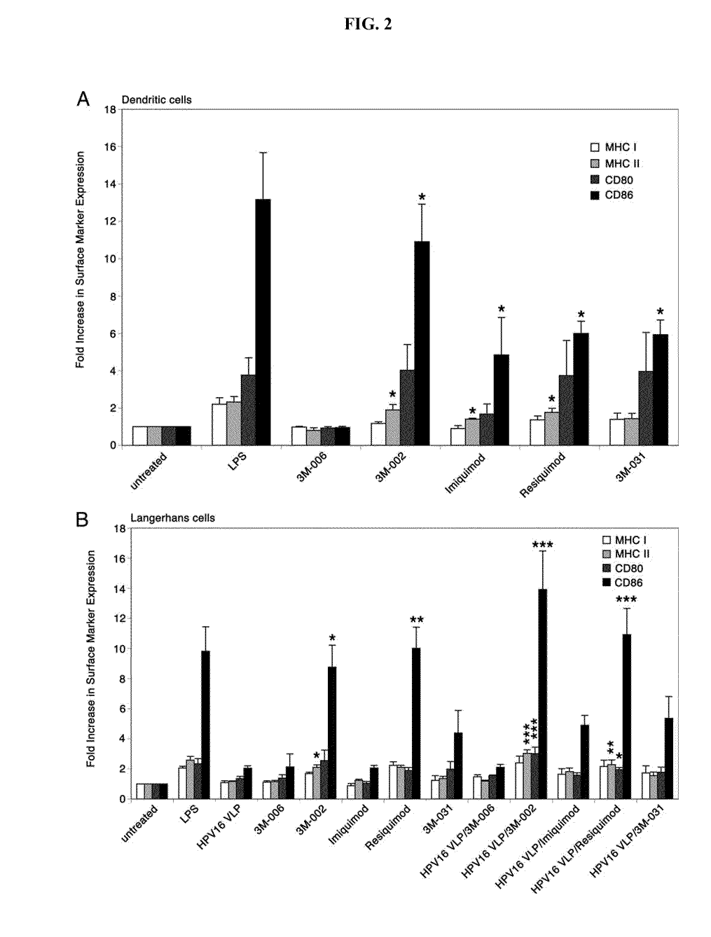

Knowing that TLR7 and TLR8 are expressed in immature LC and LC exposed to HPV16 VLP, Applicants sought to determine whether selected synthetic imidazoquinolines phenotypically activate LC exposed to HPV16 VLP. Applicants assessed phenotypic activation by the expression of surface markers, MHC class I, MHC class II, CD80, and CD86, on LC that have previously encountered HPV16 VLP and have been treated with each of the imidazoquinolines. DC, which are potent professional APC that reside within the dermis, were used as a positive control test for the activity and to determine the optimal concentration of each imidazoquinoline because it has been well established that DC are activated by imidazoquinoline compounds (Sauder (2003) Br. J. Dermatol. 149: 5-8, 34, 35; Stanley (2002) Clin. Exp. Dermatol. 27: 571-577; and Philbin and Levy (2007) Biochem. Soc. Trans. 35: 1485-1491). As expected, DC treated with 3M-002, imiquimod, resiquimod, and 3M-031 induced the up-regulation of surface markers, most notably MHC class II and CD86, relative to untreated or 3M-006-treated DC (FIG. 2A).

3M-006 is an inactive small molecule TLR7/8 analog that is produced in a similar manner as the other imidazoquinolines and used as a negative control. The optimal concentration for each imidazoquinoline to activate APC was determined by assessing a range of concentrations (0.1-60 μM) for each agonist. The concentration of each agonist that resulted in the maximum expression of surface makers on DC, as determined by flow cytometry analysis, was used as the optimal concentration (data not shown). Since Applicants confirmed that the agonists are active and knowing the optimal concentrations needed to activate APC, Applicants investigated whether each agonist has the ability to reverse the phenotype of LC exposed to HPV16 VLP. LC were left untreated, stimulated with LPS, exposed to HPV16 VLP, treated with each of the imidazoquinolines, or exposed to HPV16 VLP and subsequently treated with each of the imidazoquinolines. Each population of cells was harvested after the final incubation and analyzed by flow cytometry for the expression of surface markers.

Consistent with the previously reported data (Fausch et al. supra), LC exposed to HPV16 VLP did not increase the expression of surface markers when compared with untreated LC and 3M-006-treated LC (FIG. 2B). LC treated with either 3M-002 or resiquimod significantly induced the up-regulation of surface marker, as seen with the positive control LPS stimulation. Surprisingly, imiquimod- and 3M-031-treated LC induced only a minor up-regulation of surface markers above that of the negative controls, untreated LC, LC exposed to HPV16 VLP, and

3M-006-treated LC (FIG. 2B). It should be noted that imiquimod could not be used at any higher dose because it was found to be toxic to the cells at 2-fold higher concentrations than used in the assays. Consequently, when LC were exposed to HPV16 VLP and subsequently treated with each of the imidazoquinolines, only 3M-002 and resiquimod significantly induced the up-regulation of the surface markers, while imiquimod and 3M-031 moderately increased the expression of surface markers on LC exposed to HPV16 VLP, relative to the negative controls (FIG. 2B). Of note, it appears that TLR7 and TLR8 agonists induced a slightly greater up-regulation of surface markers on LC that have previously been exposed to HPV16 VLP than on untreated LC; however, these differences in expression are not statistically significant. Thus, these phenotypic data begin to suggest that imidazoquinolines have different effects on DC and LC. Specifically, 3M-002 and resiquimod appear to be far more potent agonists for LC than imiquimod and 3M-031.

Differential Production of Cytokines and Chemokines from LC Stimulated with Imidazoquinolines

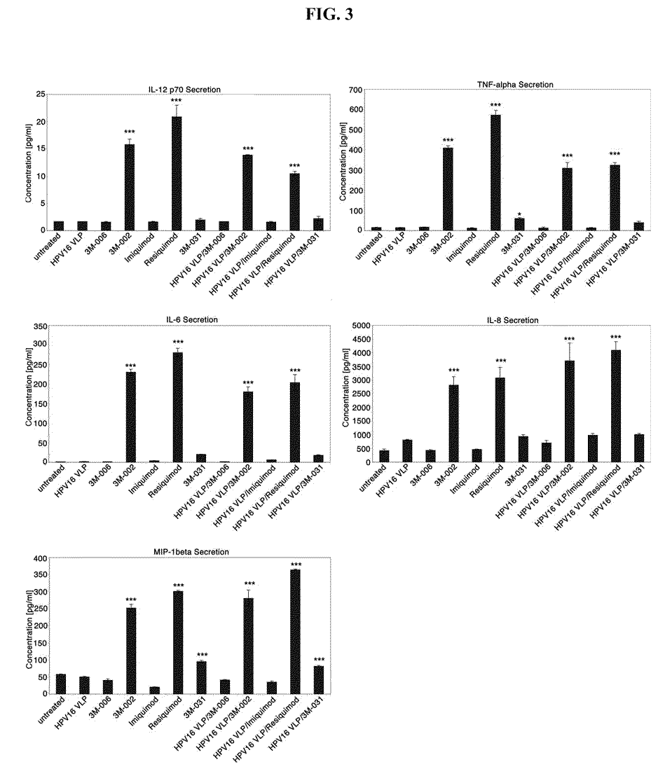

Imidazoquinolines stimulate both an innate and an adaptive immune response. The innate immune response induced by imidazoquinolines drives the adaptive immune response into a Th1 cell-mediated response via the local cytokine and chemokine milieu generated primarily by activated macrophages and DC. Thus, Applicants wanted to determine whether selected imidazoquinolines could stimulate LC exposed to HPV16 VLP to produce a proinflammatory cytokine and chemokine profile similar to the cytokine milieu known to be generated by imidazoquinoline-activated DC. Cytokines and chemokines produced by untreated LC, LC exposed to HPV16 VLP, LC treated with each of the imidazoquinoline compounds, and LC exposed to HPV16 VLP and treated with the imidazoquinoline compounds were evaluated. Supernatant from each treatment was collected and analyzed using a human cytokine LINCOplex assay. IL-12p70, TNF-α, IL-6, IL-8, and MIP-1β concentrations were statistically significantly elevated when LC were stimulated with 3M-002, resiquimod, or when LC were exposed to HPV16 VLP and then stimulated with either 3M-002 or resiquimod in comparison to the negative controls, untreated LC, LC exposed to HPV16 VLP, 3M-006-treated LC, and LC exposed to HPV16 VLP and treated with 3M-006 (FIG. 3). LC treated with 3M-031 or LC exposed to HPV16 VLP and subsequently stimulated with 3M-031 only slightly induced the production of these cytokines and chemokines above that of the negative controls (FIG. 3). IP-10, MCP-1, and RANTES (CCL5) were also found to be highly secreted by LC treated with 3M-002, resiquimod, or 3M-031 and LC exposed to HPV16 VLP and then stimulated with either 3M-002, resiquimod, or 3M-031 (data not shown). Markedly, imiquimod-stimulated LC and LC exposed to HPV16 VLP and subsequently treated with imiquimod secreted comparable amounts of TNF-α, IL-12p70, IL-6, IL-8, MIP-113 (FIG. 3), IP-10, RANTES, or MCP-1 (data not shown) as that observed in the negative controls. The cytokine and chemokine analyses demonstrate that 3M-002 and resiquimod are more efficient activators of HPV16 VLP-exposed LC in comparison to 3M-031 and imiquimod. The cytokine and chemokine profiles produced by both 3M-002- and resiquimod-activated LC are similar to those of imidazoquinoline-stimulated DC (Sauder, D. N. supra and Stanley M. A. supra). Thus, like DC, LC activated by either 3M-002 or resiquimod likely induce a Th1 cell-mediated response via the production of cytokines and chemokines.

### 3M-002 and Resiquimod Induce the Up-Regulation of CCR7 and Migration of LC Exposed to HPV16 VLP Toward CCL21

In addition to the up-regulation of surface markers and the secretion of proinflammatory cytokines and chemokines, another hallmark of LC activation is the up-regulation of CCR7 and the migration out of peripheral tissues toward draining LN. CCR7 mediates the migration of LC to T cell zones of the draining LN by binding to either secondary lymphoid tissue chemokine (secondary lymphoid tissue chemokine/CCL21) or MIP-3β (CCL19).

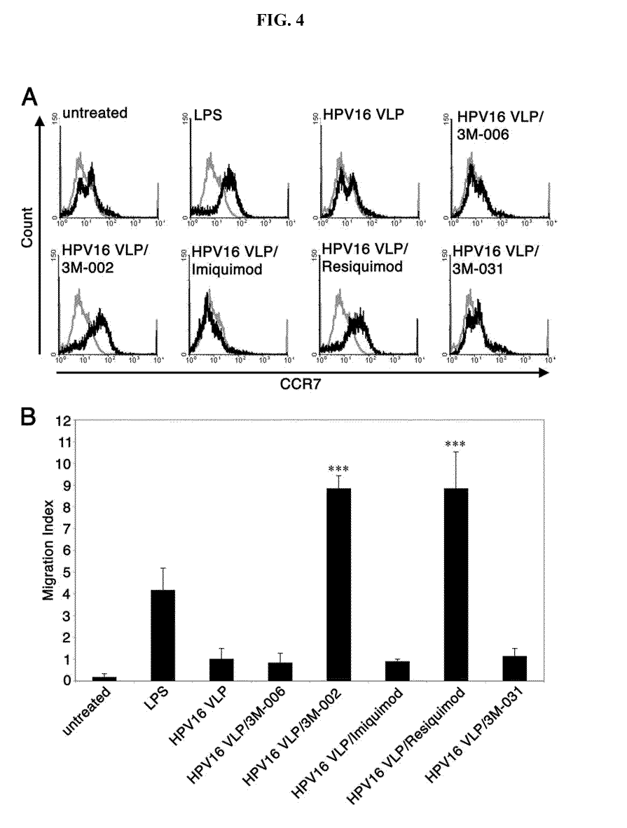

Therefore, Applicants investigated whether the imidazoquinoline compounds can induce the up-regulation of CCR7 and CCL21-directed migration of LC exposed to HPV16 VLP. Untreated LC, LPS stimulated LC, LC exposed to HPV16 VLP, and LC exposed to HPV16 VLP and subsequently treated with each of the imidazoquinolines were analyzed for the expression of CCR7 by flow cytometry. LC exposed to HPV16 VLP stimulated with either 3M-002 or resiquimod induced the up-regulation of CCR7 similar to the positive control LPS-treated LC (FIG. 4A). In contrast, imiquimod and 3M-031 did not induce the expression of CCR7 on LC previously exposed to HPV16 VLP (FIG. 4A).

Next, Applicants examined whether the expression of CCR7 functionally corresponded to enhanced migration of LC toward CCL21 by a Transwell migration assay. Applicants observed that 3M-002 and resiquimod significantly induced the migration of LC exposed to HPV16 VLP toward CCL21, as seen similarly in the positive control, while imiquimod and 3M-031 did not enhance CCL21-directed migration of LC exposed to HPV16 VLP (FIG. 4b). Taken together, these experiments demonstrate that 3M-002 and resiquimod are providing LC exposed to HPV16 VLP with a potent stimulus to acquire the potential to migrate effectively in response toward a LN-derived chemokine, CCL21.

Induction of an Epitope-Specific CD8⁺ T Cell Response by LC Exposed to HPV16 cVLP and Stimulated with Either 3M-002 or Resiquimod

Thus far, Applicants have demonstrated that 3M-002 and resiquimod can effectively activate LC previously exposed to HPV16 VLP, unlike imiquimod and 3M-031; therefore, Applicants next sought to determine whether LC exposed to HPV16 VLP and stimulated with each of the imidazoquinolines could induce an HPV16-specific, MHC class I-restricted T cell response by performing in vitro immunization assays. HPV16 cVLP were used in these experiments because they contain a well-characterized human HLA-A*0201-restricted epitope (E7 peptide aa 86-93, TLGIVCPI) recognized by human CD8⁺ T cells (Ressing, M. E et al. supra). Human DC, but not LC, have been shown to initiate epitope-specific responses to this peptide when exposed to the HPV16 cVLP (Fausch supra and Rudolf et al. supra). Thus, HPV16 cVLP were used to determine whether the imidazoquinoline compounds are capable of stimulating LC exposed to HPV16 VLP to initiate an epitope-specific immune response against the HPV16 E786-93 peptide.

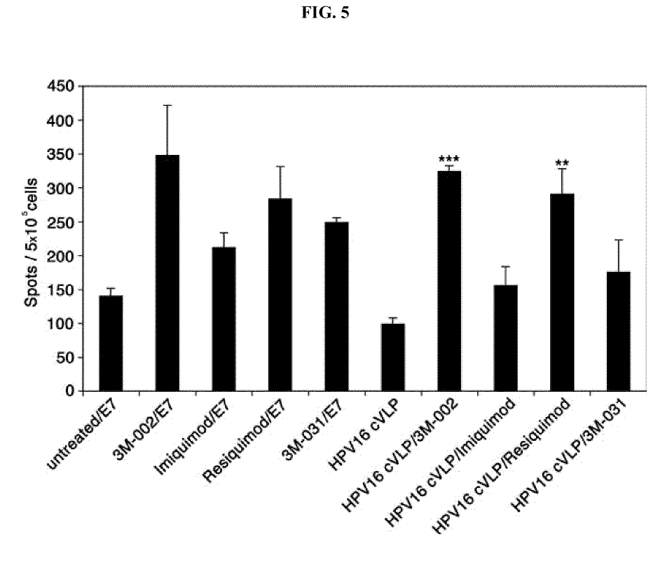

In the experiments presented here, LC generated from HLAA* 0201-positive monocytes were exposed to HPV16 cVLP and treated with each of the imidazoquinolines. Applicants then incubated the cells with autologous naive CD8⁺ T cells and the cultures were stimulated twice with their respective treated LC. As control treatments LC were treated with each of the imidaziquinolines and pulsed with the HPV16-E7-derived HLA-A*0201-restricted CTL epitope (E786-93). Seven days after the last re-stimulation, the cells from each culture were collected and analyzed for a specific CD8⁺ T cell response to the HLA-A*0201-restricted HPV16-E786-93 peptide by an IFN-γ ELISPOT. Of major impact, LC exposed to HPV16 cVLP and stimulated with either 3M-002 or resiquimod initiated a statistically significant HPV16 epitope-specific response when compared with untreated LC and LC exposed to HPV16 cVLP, while LC exposed to HPV16 cVLP and stimulated with either imiquimod or 3M-031 did not induce a significant HPV16 epitope-specific immune response (FIG. 5). Collectively, these experiments demonstrate that both 3M-002 and resiquimod effectively induce LC activation and have the ability to initiate an HPV16-specific cell-mediated immune response through the activation of LC.

In this study, Applicants investigated synthetic imidazoquinolines as potential activators of LC previously exposed to HPV16 VLP, which could lead to further exploration of specific imidazoquinolines as therapeutic compounds for treating existing HPV16-induced cervical lesions. The data clearly demonstrate that 3M-002 and resiquimod can induce the phenotypic maturation of naive LC and LC previously exposed to HPV16 VLP via the up-regulation of surface markers (MHC class I, MHC class II, CD80, and CD86). Moreover, 3M-002 and resiquimod induce functional activation of LC exposed to HPV16 VLP as demonstrated by the production of Th1-associated cytokines and chemokines, CCL21-directed migration, and the induction of an HPV16-specific CD8⁺ T cell response. However, imiquimod does not phenotypically or functionally activate LC while 3M-031 partially induces the activation of LC. Collectively, the data suggest that 3M-002 and resiquimod can reverse the phenotype and function of LC exposed to HPV16, unlike imiquimod and 3M-031. Therefore, the results support exploring 3M-002 and resiquimod as therapeutic small-molecule compounds for treating HPV infections and HPV-induced cervical lesions.

The findings presented here are based upon a model system that mimics the interaction between HPV and LC in the human epidermis. Monocyte-derived LC are an appropriate alternative model, because they express MHC class II molecules, langerin, E-cadherin, CD1a, and Birbeck granules (Fausch et al. supra), which classically define human LC located in the epidermis (Merad et al. (2008) Nat. Rev. Immunol. 8: 935-947). Recently, the status of LC as the only APC in the epithelium that express langerin was challenged. It was reported that dermal langerin⁺ DC exist in mice and may play a role in the immunosurveillance of the skin (Poulin et al. (2007) J. Exp. Med. 204: 3119-3131 and Ginhoux et al. (2007) J. Exp. Med. 204: 3-3146). However, Klechevsky et al. (Klechevsky et al. (2008) Immunity 29: 497-510) demonstrated that although two different subsets exist of human dermal DC, neither of these subsets express langerin, highlighting a difference in human and murine APC populations located in the epithelium (Klechevsky et al. supra). Moreover, this study was conducted using VLP, which have been developed as an alternative to HPV virions for immunological analysis. This is because the life cycle of HPV is dependent on the differentiation of cells in the squamous epithelium, making it difficult to produce large quantities of HPV virions in vitro.

Thus, due to the facts that human LC are the only APC at the site of infection, that monocyte-derived LC have been shown to be phenotypically equivalent to human epidermal LC, and that VLP are an accepted alternative to purified virions for immunological analysis of HPV, this study uses the most appropriate model to critically examine the interaction of HPV and human LC. Imiquimod is a Food and Drug Administration-approved drug (Aldara) to treat external anogenital warts (condyloma accuminatum) caused by low-risk HPV infection (HPV 6 and 11). More recently, imiquimod has been shown to be successful in treating high-risk HPV-induced vulvar intraepithelium neoplasia (VIN) (van Seters et al. (2008) N. Engl. J. Med. 358(41): 1465-1473). However, imiquimod has yet to be reported as an effective therapeutic treatment for HPV-induced cervical intraepithelium neoplasia. The reason why there is a difference in response initiated by imiquimod against different types of HPV-induced lesions (genital warts, VIN lesions, and cervical intraepithelium neoplasia lesions) is unclear.

This disparity in response could be due to the difference in cellular composition and structure of the external genitalia and the cervix. Considering, Applicants demonstrate that imiquimod does not activate LC, an effective immune response against anogenital warts and VIN lesions is likely due to the activation of APC other than LC, such as DC and macrophages.

The effects of synthetic imidazoquinolines on LC had not been well studied until now. Previously, it was shown that imiquimod and resiquimod do not phenotypically but functionally activate LC (Burns et al. (2000) Clin. Immunol. 94: 13-23 and Suzuki et al. (2000) J. Invest. Dermatol. 114: 135-141). Past studies assessed phenotypic activation of LC by the expression of surface markers. The results from these studies are in accordance with the results for imiquimod; however, Applicants found that resiquimod does phenotypically activate LC. The reason for this discrepancy between the present and past studies, concerning the effects of resiquimod on LC, could be explained because Burns et al. examined phenotypic activation 6 h after LC were treated with resiquimod, while Applicants assessed the maturation of LC 24 h after treatment with resiquimod. Furthermore, the functional activation of LC was examined in the previous studies in multiple ways, one of which was by the level of mRNA-encoding proinflammatory cytokines, such as TNF-α, IL-6, and IL-12p40.

The results from these studies showed that imiquimod and resiquimod enhanced the transcription of the genes for these specific cytokines. Applicants also assessed cytokine levels as a means of evaluating functional activation; however, Applicants did so at the more relevant level of protein production. Applicants observed proinflammatory cytokine and chemokine secretion by LC treated with either 3M-002 or resiquimod and to a modest extent with 3M-031; however, Applicants did not observe this with imiquimod. The results are different from previous reports because Applicants assayed for a different end product, namely, protein, and mRNA transcripts do not always translate to protein expression. Additionally, the results are consistent with recent findings showing that TLR8 agonists are more effective than TLR7 agonists at inducing proinflammatory cytokines and chemokines by monocyte-derived DC (GM-CSF/IL-4/TGF-13) (Gorden et al. (2005) J. Immunol. 174:1259-1268). During activation LC migrate out of the epidermal tissue to draining LN where they activate naive T cells, thereby inducing a cell-mediated immune response. Previous data have demonstrated that LC exposed to HPV16 VLP cannot up-regulate CCR7, migrate, or induce an HPV16-specific CD8⁺ T cell response (Fausch et al. (2002) J. Immunol. 169:3242-3249 and Fausch et al. (2005) J. Immunol. 174: 7172-7178). To explore the effects of synthetic imidazoquinolines on the migration of LC exposed to HPV16 VLP, Applicants assessed the expression of CCR7 and the ability of LC previously exposed to HPV16 VLP to migrate toward CCL21.

The results clearly show CCR7 is up-regulated on LC exposed to HPV16 VLP that are treated with either 3M-002 or resiquimod, but not when treated with imiquimod or 3M-031. Furthermore, Applicants demonstrate that the expression of CCR7 correlates to the migratory ability of LC exposed to HPV16 VLP. The data illustrate that only 3M-002- and resiquimod-treated LC previously exposed to HPV16 VLP are able to migrate in response to CCL21. However, in a contrasting study, it was shown that imiquimod functionally activates LC by demonstrating that imiquimod induces the migration of LC, yet the study was performed using a mouse model and it was not confirmed that the migrating LC were effective in inducing an epitope-specific adaptive immune response (Suzuki et al. (2000) J. Invest. Dermatol. 114:135-141).

Nevertheless, Applicants sought to determine whether LC exposed to HPV16 VLP that are treated with imidazoquinolines have the ability to induce an HPV16 epitope-specific CD8⁺ T cell response. The results show that 3M-002 and resiquimod can effectively overcome the phenotype and function of LC exposed to HPV16 VLP and can induce an HPV16-specific CD8⁺ T cells response, which is critical in mediating the clearance of HPV16 infections and HPV16-induced cervical lesions. In addition to the findings, Burns et al. (Burns et al. (2000) Clin. Immunol. 94: 13-23) investigated the functional activation of LC after treatment with either imiquimod or resiquimod by assessing the allostimulatory capacity of the treated LC. They found that imiquimod only modestly induced T cell proliferation in an allogenic MLR assay while resiquimod highly increased the allostimulatory capacity of LC (Burns et al. (2000) Clin. Immunol. 94: 13-23). Their results from this functional assay are in line with the functional data, which is further support that resiquimod is more potent than imiquimod in activating LC.

Collectively, the findings imply that strong TLR8 agonists, such as 3M-002 and resiquimod, are more effective in inducing LC activation and overcoming the tolerizing-like phenotype and function of LC exposed to HPV16 VLP, in comparison to TLR7 agonists, such as imiquimod. It has been shown that TLR7 and TLR8 agonists differ in their target cell selectivity (Gorden et al. (2005) J. Immunol. 174:1259-1268). Notably, resiquimod and 3M-031 are both TLR7 and TLR8 agonists; however, resiquimod is much more effective in activating LC. This may occur because the agonists differ in their target cell selectivity and preferentially activate one TLR over the other; resiquimod is known to preferentially act through TLR8 (Scho\ddot{}n and Scho\ddot{}n (2008) Oncogene 27: 190-199), while it has yet to be reported which receptor 3M-031 preferentially acts through.

This explanation is plausible considering that functional differences have been observed between TLR7 and TLR8 (Gorden et al. (2005) J. Immunol. 174(44):1259-1268). It was demonstrated that TLR7 activation primarily leads to the production of IFN-γ- and IFN-regulated cytokines, which is similar to TLR9 activation, while TLR8 is functionally associated with the production of proinflammatory cytokines, such as TNF-α (Gorden et al. supra). One explanation for the functional distinction between TLR7 and TLR8 is the difference in the signal transduction pathways initiated by each of the receptors. TLR8-mediated activation of NF-κB and JNK are dependent on MEK kinase 3 (Qin et al. (2006) J. Biol. Chem. 281: 21013-21021), while TLR7-mediated activation of NF-κB is TGF-β-activated kinase 1 dependent (Agrawal and Kandimalla (2007) Biochem. Soc. Trans. 35: 1461-1467).

Bruton tyrosine kinase has also been shown to directly interact with the intracellular domain of TLR8 and plays an important role in the signal transduction of TLR8, yet Bruton tyrosine kinase has not been demonstrated to be associated with TLR7 (Jefferies et al. (2003) J. Biol. Chem. 278: 26258-26264 and Sochorova’ et al. (2007) Blood 109: 2553-2556). Alternatively, another explanation of the findings may be that TLR8 is inhibiting TLR7 function. In HEK293 cells, it was demonstrated that the coexpression of TLR8 and TLR7 results in inhibition of TLR7 to respond to its agonist (Wang et al. (2006) J. Biol. Chem. 281: 37427-37434). Therefore, TLR8 may inhibit LC from responding to agonists that preferentially bind TLR7, which explains why TLR8-dominant agonists (such as 3M-002 and resiquimod) are more effective than TLR7-dominant agonists (such as imiquimod and potentially 3M-031) in activating LC and in driving a strong cell-mediated immune response.

Since LC are critical in controlling the induction of an immune response in the epithelium and they are targeted by HPV16 to escape immune detection, LC are attractive targets for immunotherapy of HPV16-induced cervical lesions. In addition, LC have recently been shown to be able to directly kill cervical epithelial cells that express HPV16 E6 and E7, thereby generating a source of Ag that could be processed and presented by APC to T cells. LC cytotoxicity is mediated in part by TRAIL expression, which can be up-regulated by the presence of IFN-γ (Le Poole et al. (2008) Cancer Immunol. Immunother. 57: 789-797). Furthermore, it has been demonstrated that TLR7/8-stimulated DC-like cells have cytotoxic activity, which is mediated by the expression of TRAIL and the secretion of perforin and granzyme B (Stary et al. (2007) J. Exp. Med. 204: 1441-1451). Thus, it is conceivable that TLR8 agonists stimulate LC not only to induce an HPV-specific Th1-mediated cellular immune response but may also enhance LC cytotoxicity toward HPV16-infected epithelial cells, further augmenting antiviral and antineoplastic activity. In conclusion, TLR8 agonists are promising therapeutic compounds for the treatment of HPV infections and HPV-induced cervical lesions.

### Example 2

### TLR Agonists Up-Regulate SLPI Production by LC and Induce HPV16-Exposed LC to Activate HPV16-Specific T Cells from Patients Infected with HPV or Co-Infected with HPV/HIV

Activation of antigen-presenting cells (APC), like LC, is required for successful interaction with and activation of primary T lymphocytes. Since LC are the only APC that contact mucosal HPV in the vaginal tract and other anogenital sites, activating HPV-exposed LC may be a step necessary to initiate an adaptive immune response that can clear mucosal HPV infection. LC express a variety of TLR such as TLR 3, 7 and 8, which are known to participate in bridging antiviral innate immunity with adaptive immunity, while epithelial cells express TLR3. TLR agonists have the potential to influence the immune-stimulatory capacity of LC and epithelial cells, if applied topically to immune-suppressed HPV-infected epithelium. The TLR7 agonist, Imiquimod, is FDA-approved for genital warts and is used off-label for other skin diseases, but is toxic to the cervix and has not led to regression of cervical intraepithelial neoplasia (CIN) in clinical trials.

Applicants previously have shown that treatment of HPV16-exposed LC with agonists to TLR8 (Resiquimod and CL075), but not TLR7 (Imiquimod), overcomes HPV16-mediated immune suppression of LC (Fahey et al. (2009) J. Immunol. 182:2919-2928). These results may explain the failure of Imiquimod to successfully treat HPV-associated lesions of the cervix. Poly-ICR is a more stable derivative of Poly-IC and is available for clinical development. These immune modulating agents have the potential to reverse HPV immune escape through activating HPV-exposed LC and therefore need to be tested and compared with previously tested compounds. Applicants' studies can define whether Poly-ICR is able to reverse the immune suppression by HPV, similar to TLR8 agonists (Fahey et al. supra). So far, none of the TLR agonists have been tested on LC from women with CIN3 or from women who are HIV⁺ and have abnormal cervical lesions. As they have developed HPV-related disease and may be immune-compromised, the immune cells (both LC and T cells) can be studied from patients that may respond differently than those of a healthy person. Applicants contemplate that positive data obtained in this aim can form the basis for testing the efficacy of TLR agonists in clearing persistent HPV infections in HIV+ individuals in near term clinical trials.

Secretory leukocyte protease inhibitor (SLPI) is present in cervicovaginal secretions and can be produced by multiple cell types. Applicants contemplate that treating HPV16-exposed epithelial cells with TLR agonists may induce the production of anti-virals and inflammatory cytokines that have the ability to contribute to activation of antigen-specific T cells, which would be important for clearance of virus infection and prevention of future lesion development.

The potency of the TLR3 agonist (Poly-ICR), TLR8 agonists (CL075), TLR 8/7 agonist (Resiquimod), and TLR7 agonist (Imiquimod) in altering production of SLPI by LC and epithelial cells, and in reversing HPV suppression of LC function is compared using cells from healthy individuals and individuals already exposed to HPV or HPV/HIV. The TLR 7 and 8 agonists are commercially available from InvivoGen. Poly-ICR is available from Nventa Biopharmaceuticals. HLA-A*0201-positive healthy donors are recruited from in or around the USC Health Science Campus for donation of leukapheresis material in accordance with an Institutional Review Board approved protocol. Pathology-confirmed HLA-A*0201⁺ CIN3⁺ patients and HIV⁺ HLA-A*0201⁺ CIN3⁺ patients are recruited from the LAC+USC medical center hospital for donation of leukapheresis material.

HLA-A*0201-positive subjects is chosen so that well defined T cell immune responses can be measured against known HPV-derived peptide antigens. Recruited patients who are HIV⁺ can be on highly active antiretroviral therapy (HAART) and must have CD4+ T cell counts >500 cells/mm³, so that an adequate T cell response can be assessed. In all experiments, LC is generated from monocytes of healthy donors, from CIN3⁺ patients, and from HIV⁺CIN3⁺ patients. Up-regulation of MHC and co-stimulatory molecules, cytokine and chemokine secretion, SLPI production and increased migration towards chemoattractive cytokines is assessed after LC are exposed to HPV16 L1L2 VLP and subsequently treated with the TLR agonists ranging from 1-100 μM by flow cytometry, multiplex cytokine assays, ELISA assays, and an in vitro transwell migration assay as described previously (Fahey et al. supra). Untreated LC is used as a negative control to set background and LPS-treated LC is used as a positive control. If HPV-exposed LC are activated by TLR agonists in the above assays, the ability of treated LC to stimulate HPV-specific T cell responses be IFN-γ is measured by Elispot to quantify HPV16-specific CD8⁺ T cell responses to L1₃₂₃₋₃₃₁ and E7₁₁₋₂₀ and T cell proliferation is measured using a standard radioactive ³H-thymidine proliferation assay. To determine whether treatment with TLR agonists also modulate SLPI and cytokine production by epithelial cells, supernatants from HPV16 L1L2 VLP-exposed primary neonatal foreskin keratinocytes, HaCaT cells and Caski cells untreated or treated with TLR agonists is assessed by ELISA for SLPI or by multiplex ELISA for inflammatory cytokines and chemokines

Without being bound by theory, Applicants submit that SLPI is up-regulated upon activation of HPV16-exposed LC with Poly-ICR given that TLR agonists can overcome suppression of LC function imparted by HPV16. Applicants also contemplate to see increased SLPI production by HPV16-exposed epithelial cells upon treatment with both Poly-ICR, similar to trophoblasts that are exposed to a TLR3 agonist. Based on the data from healthy donors (Fahey et al. supra), Applicants contemplate that Resiquimod, CL075 and but not Imiquimod, phenotypically and functionally activate human LC from CIN/HIV patients. Applicants also contemplate that Poly-ICR activates human LC from both healthy donors and CIN/HIV patients, though one may be superior to the other. Without limited by theory, Applicants contemplate that increased levels of MHC and surface activation molecules, increased secretion of inflammatory and T cell activating cytokines and chemokines, and an increased ability to perform chemokine-directed migration after HPV and TLR agonist treatment would all support the hypothesis.

Applicants also contemplate that triggering TLR3 or TLR8 with Poly-ICR treatment results in the activation of HPV peptide-specific T cells, thus overcoming HPV-induced tolerization of LC. Applicants contemplate if TLR agonists enable HPV-exposed LC to gain back their T cell immune-stimulatory capacity and migrate—both functions needed for a productive anti-viral immune response. Importantly, these data indicate whether HPV-induced immune escape can be reversed in HPV-infected patients who may have developed peripheral tolerance towards HPV and in HIV/HPV-infected patients who might be slightly more immune compromised.

### Example 3

### HPV16 and Other High-Risk and Low-Risk HPV also Suppress LC Maturation, and Treatment of LC with TLR Agonists Reverses the Immune Escape of Other High Risk HPV Genotypes that can Cause Cancer in HIV-Infected Individuals

The L2 protein of mucosatropic papillomaviruses is highly conserved; however it is unknown whether the immune suppressive effect of HPV L1L2 particles is conserved amongst the high-risk, low-risk, mucosal and skin-tropic viruses. Applicants contemplate to analyze genotypes from each of these papillomaviral classes to determine whether HPV effects on LC maturation and function differ depending on how likely the virus is to cause neoplastic disease. Several studies have found a broader range of HPV types in HIV-positive compared with HIV-negative women, as well as more concomitant infections frequently detected in high grade lesions (De Vuyst et al. (2008) Eur. J. Cancer Prey. 17:545-554).

With HPV16 accounting for a smaller proportion of HPV infections in HIV+ women, the challenge is to develop an HPV therapeutic that is not specific for any one HPV genotype. Without limited by any theory, Applicants contemplate that the concept of reversing HPV immune escape through TLR activation of LC is not genotype-specific, but may require that all the high-risk genotypes utilize this same mechanism of immune escape. Applicants contemplate that the data obtained in this aim will provide information on how widespread the effect of HPV suppression is on LC function, and whether the suppression is conserved amongst HPV genotypes based on viral classifications and the specific interactions of the different L2 proteins with LC. If all the high risk genotypes (and low-risk genotypes) are shown to suppress LC function and the effects are mediated through ANXA2, then its contemplated that the strategies developed as a result of this proposal will be applicable to any HPV infection which is important especially for the population of HIV-infected individuals that are at a higher risk for developing HPV-associated diseases.

In order to determine whether other genotypes also suppress LC function, Applicants use pseudovirions (VLP containing genetic material produced in mammalian cells) or VLP (empty capsids produced in insect cells) from several additional genotypes and analyze LC for phenotypic maturation, cytokine and chemokine secretion, and migration as described earlier. A comparison of the effects of L1 versus L1L2 VLP on LC activation is performed to determine whether the L2 proteins of other genotypes participate in immune modulation. Applicants use HPV1 to represent the skin wart causing genotype, HPV11 to represent low-risk mucosal genotype, and HPV18, 31, 45, and 58 to represent additional high-risk genotypes. CD8⁺ T cell epitopes from the capsid proteins have not been identified, except for HPV16. The sequences are divergent enough such that an immunogenic epitope for one will not be the same in the others. As an alternative to capsid epitopes, Applicants treat LC with the different HPV genotypes and the HLA-A*0201 HPV16 E7₁₁₋₂₀ peptide (Ressing et al. (1995) The Journal of Immunology 154:5934-5943), with or without CD40L to activate the LC, to assess CD8+ T cell responses by IFNγ Elispot and proliferation assays as described. To determine whether the TLR agonists can activate the high- and low-risk HPV-exposed LC similar to HPV16-exposed LC, Applicants perform phenotypic and functional LC activation assays as described in Example 2 by first exposing LC to the HPV genotypes, then treating cells with TLR agonists, followed by analysis for activation, cytokine/chemokine production and chemokine-directed migration.

Based on the preliminary data with HPV18 L1 and L1L2 VLP, in combination with published studies that high-risk HPVs have longer persistence periods, Applicants contemplate that the high-risk HPVs 18, 31, 45 and 58 will suppress LC more than the cutaneous genotypes HPV1 and 5 and less than the low-risk HPV11 genotype. If all genotypes similarly fail to activate LC, without limited by any theory, it is contemplated that these results would indicate that all HPVs, no matter what classification, have evolved to evade immune detection by suppressing LC and this may be a normal part of their pathogenesis, unrelated to the type of lesion most associated with that genotype. If the L2 protein of all genotypes is involved in the mechanism of immune escape through LC interaction, then Applicants contemplate that like HPV16, the HPV1 L1L2, HPV11 L1L2, and HPV18 L1L2 will suppress LC maturation and function, whereas the L1 VLP counterpart to each genotype will activate LC. Without limited by any theory, Applicants further contemplate that a negative result would suggest that the immune regulatory function of HPV16 L2 is unique only to that genotype, which could explain why it is considered the most oncogenic of all the HPV types and why it is the most frequently found genotype in cervical neoplasia, regardless of geography and other environmental factors. However since Applicants have observed similar results with HPV18, Applicants view this as unlikely and attribute the increased oncogenicity of HPV16 to other intrinsic factors once it has integrated into the host genome.

### Example 4

### Statistical Analysis

All experiments described in Examples 2 and 3 can be repeated several times to ensure reproducibility and statistical significance. Parametric and non-parametric statistical techniques can be used, when appropriate, to avoid assumptions regarding distributions of the data. Differences in immunologic response measures can be determined using a one-way analysis of variance (ANOVA, parametric) or Kruskal-Wallis test (nonparametric) for analyzing overall between group differences for each outcome variable. Student's t tests (parametric) or Mann-Whitney U tests (nonparametric) can be used for between groups' statistical analyses. In all cases, a two-sided alpha level of 0.05 can be considered statistically significant. Significance levels can be adjusted using the Bonferroni or Tukey method as appropriate when multiple statistical analyses are performed.

### Example 5

### Reversal of HPV Immune Suppression with TLR Agonists

Since TLR expression on LC was not well characterized, Applicants analyzed TLR expression and found that monocyte-derived LC express TLR3, TLR7, and TLR8, suggesting that the cells may respond to their agonists (data not shown). To determine whether TLR agonists can reverse the immune suppression by HPV16 on LC, Applicants compared the efficacy of Imiquimod (TLR7 agonist), Resiquimod (TLR8/7 agonist) and CL075 (TLR8 agonist).

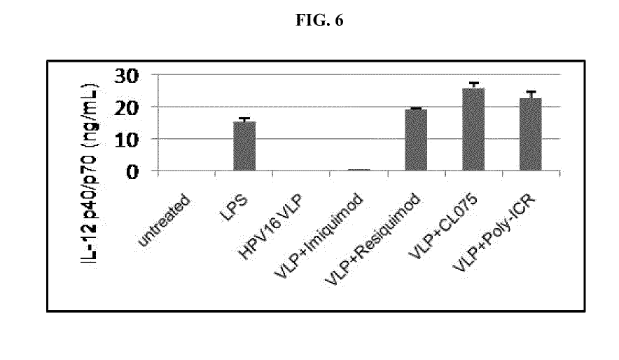

LC were left untreated or exposed to HPV16 L1L2 VLP for 18 h at 37° C. Subsequently, the cells were treated with LPS, Imiquimod, Resiquimod, CL075 or Poly-ICR for 24 h at 37° C. Cell supernatants were analyzed for IL-12 p40/p70 using a Bio-plex suspension bead ELISA (BioRad, Hercules, Calif.). FIG. 6 shows one of three representative experiments and cytokine concentration in ng/ml ±SD.

Interestingly, while Imiquimod did not activate HPV-exposed LC, the TLR 8 activators (CL075 and Resiquimod) fully activated HPV treated LC such that they up-regulated co-stimulatory and activation molecules, secreted large amounts of the Th1-promoting cytokines and activated HPV-specific T cells (Fahey et al supra). However, Resiquimod and CL075 are not available for clinical development (Pfizer Corp., personal communication). The preliminary data suggest that the TLR3 activator, Poly-ICR, can induce the production of IL-12, a Th1 promoting cytokine (FIG. 6), suggesting that it may be possible to use Poly-ICR to reverse immune suppression of LC induced by HPV16.

### Example 6

### Reversal of HPV-Mediated Immune Suppression with the TLR3 Agonist Poly-ICR

This exmple shows that the TLR3 angonist, PolyICR, was able to activate LC that had been pre-exposed to HPV16 VLP such that expression of MHC, CD40, CD80, CD86, and CD83 were highly upregulated and LC secreted high amounts of Th1 and inflammatory cytokines and chemokines. Further, upregulation of the chemokine receptor CCR7 resulted in a significant increase in migration capacity. Also, LC incubated with HPV16 VLP and treated with PolyICR induced an HPV16-specific CD8+ T cell response detected by interferon gamma Elispot and MHC tetramer analysis that was absent when LC were exposed to VLP alone.

These data indicate that the TLR3 agonist PolyICR is a promising therapeutic molecule that can overcome HPV-induced immune suppression of LC and result in an LC capable of stimulating an anti-HPV T-cell mediated immune response.

Human LC were analyzed for the expression of MHC and T-cell co-stimulatory molecules, production of Th1 inducing cytokines, in vitro migration, and activation of HPV16-specific T cells when LC were exposed to HPV16 VLP and subsequently to PolyICR.

The HPV family of viruses establishes persistent infections because it has evolved mechanisms that allow it to evade the human immune system. HPV-mediated suppression of antigen presentation by Langerhans cells (LC) is identified as a key mechanism through which HPV evades immune surveillance. PolyICR is a stable TLR3 agonist that is a broad inducer of innate immunity and is being developed as a vaccine adjuvant and antitumor agent. An important feature of PolyICR is its ability to enhance dendritic cell expression of cell surface markers, cytokine production and functional activation of T cells.

### Example 7

### Poly-ICR Induces Upregulation of MHC and Costimulatory Molecules on Human LC Exposed to HPV16

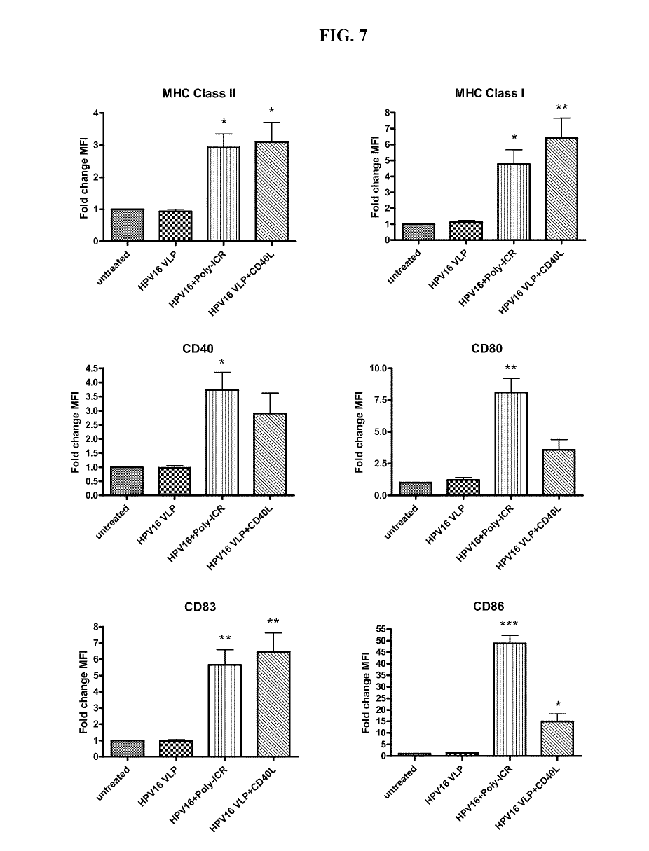

In this study, it was determined whether Poly-ICR can overcome HPV-induced immune suppression by functionally activating LC in the presence of HPV16 and inducing activation of HPV16-specific T cells. Human LC were analyzed for the expression of MHC and T-cell co-stimulatory molecules which are involved in presentation of HPV peptides and activation of CD4+ and CD8+ T cells. Poly-ICR was able to activate LC that had been pre-exposed to HPV16 VLP such that expression of the peptide presenting molecules, MHC Class I and MHC Class II, and the costimulatory molecules CD40, CD80, and CD86 were significantly upregulated (FIG. 7). The maturation marker for antigen presenting cells, CD83, was also highly upregulated after Poly-ICR treatment.

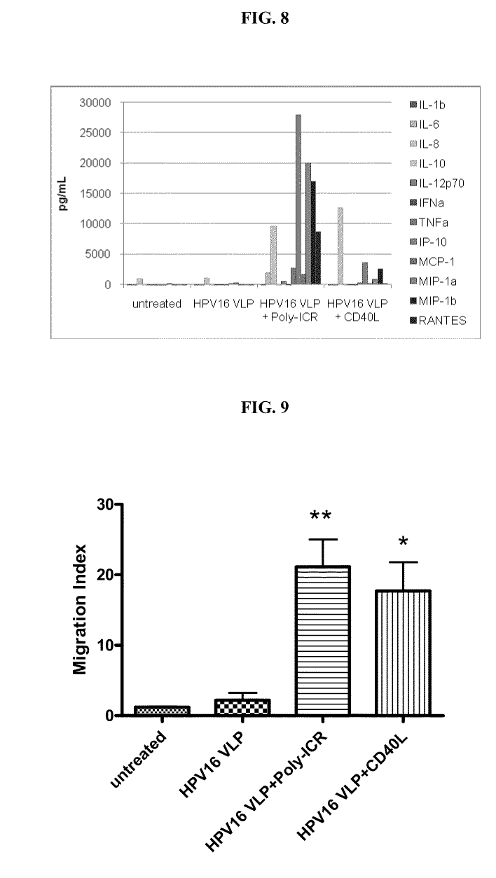

Induction of T cell responses against virus-infected cells requires antigen presenting cells to produce Th1 inducing cytokines and chemokines to prime CD8+ T cells against viral antigens. Inflammatory cytokines secreted at the site of infection also recruit innate immune cells to participate in eradication of virus-infected cells. LC were tested for the ability to secrete a wide variety of cytokines and chemokines after exposure to HPV16 followed by treatment with Poly-ICR. In contrast to CD40L, a protein involved in licensing antigen presenting cells to activate CD8+ T cells, treatment of LC with Poly-ICR resulted in an increase in both the breadth and magnitude of cytokines and chemokines produced. Among the cytokines produced by Poly-ICR treated LC were interleukin (IL)-6, IL-8, tumor necrosis factor (TNF)-a, interferon-inducible protein (IP)-10, monocyte chemo-attractant protein (MCP)-1, macrophage inflammatory protein (MIP)-1a, MIP-1b, and RANTES (FIG. 8).

LC migration to regional lymph nodes after receiving maturation signals in the periphery is required for successful interaction with naïve T cells. As an in vitro correlate of LC migration, an in vitro transwell chemotaxis assay to CCL21 was used to assess migratory capacity of LC after exposure to HPV16 followed by treatment with Poly-ICR. CCL21 is a chemokine that is expressed in lymphoid organs and signals through the maturation-induced CCR7 receptor on LC during migration to lymph nodes. Treatment of LC with Poly-ICR resulted in a significant increase in migration capacity compared to untreated LC or LC exposed to HPV16 alone (FIG. 9). Similar migration was observed with Poly-ICR and CD40L, indicating that both treatments lead to enhanced migration to CCL21.

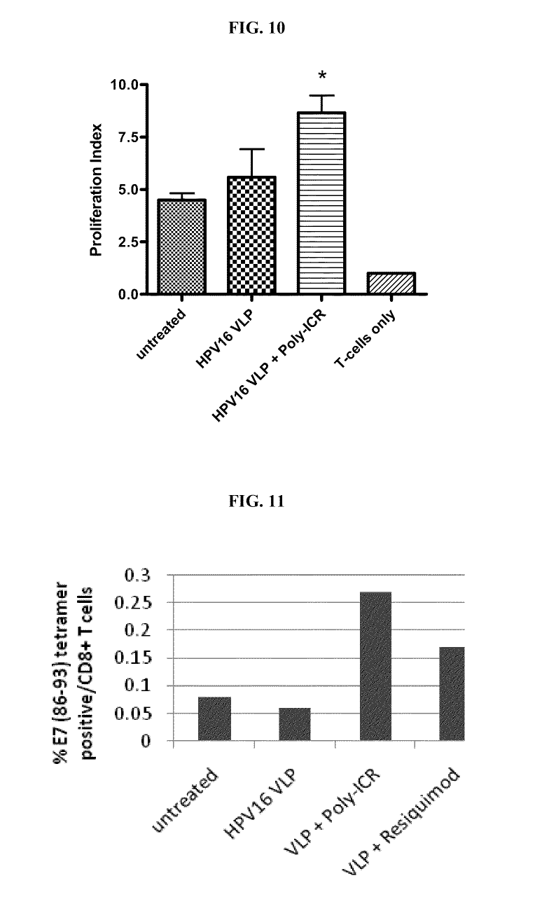

Mature activated LC are potent stimulators of T cell proliferation. To determine whether poly-ICR treatment also translated to an increased T cell stimulatory capacity of LC, a mixed lymphocyte reaction (MLR) assay was performed with HPV16-exposed Poly-ICR treated LC co-cultured with allogeneic (MHC-mismatched) purified T cells. Proliferation was measured by uptake of radioactive thymidine by proliferating T cells. Poly-ICR clearly enhanced the T cell stimulatory capacity of LC previously exposed to HPV16 VLP compared to untreated LC (FIG. 10).

### Example 8

### HPV16 VLP-Exposed LC Treated with Poly-ICR Induce an HPV16-Specific CD8+ T Cell Response

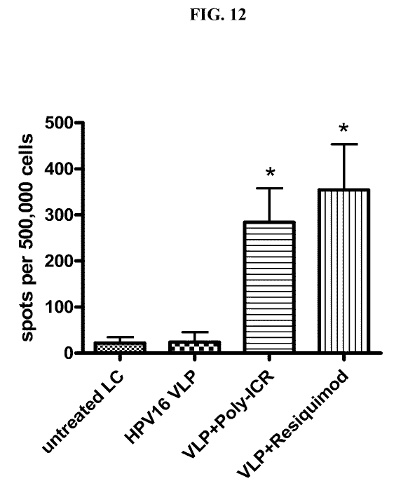

The ultimate marker of a mature and functional LC is the ability to induce antigen-specific T cells. Therefore, it was tested whether LC incubated with HPV16 VLP and treated with Poly-ICR induced an HPV16-specific CD8+ T cell response after an in vitro immunization assay. HPV16-specific T cells were quantitated by MHC tetramer analysis and interferon gamma Elispot. LC exposed to HPV16 VLP and subsequently treated with Poly-ICR were able to induce E7₈₆₋₉₃-specific CD8+ T cells when compared to untreated LC or LC exposed to HPV16 VLP alone measured my MHC class I tetramer binding (FIG. 11). LC treated with resiquimod, a TLR7/8 agonist, also induced HPV16 epitope-specific T cells. In these experiments, VLP containing the E7 protein were used and E7-binding HLA-A*0201 binding peptides were used to measure HPV-specific CD8+ T cell responses. In addition to increasing the numbers of HPV-specific CD8+ T cells, these T cells were also functionally able to secrete IFNγ in response to peptide stimulation (FIG. 12). These data indicate that the TLR3 agonist Poly-ICR is a therapeutic molecule that can overcome HPV-induced immune suppression of LC and result in an LC capable of stimulating an anti-HPV CD8+ T-cell mediated immune response.

### Example 9

### Reversal of Immune Escape in HPV-Infected Patients by the Use of Toll Like Receptor 3 Agonists

LC's expression levels of T cell co-stimulatory markers CD80, CD83, CD86 and the cell migration marker CD197 can be determined when LC are exposed to Poly-ICLC and Poly-ICR and HPV. LC are differentiated from peripheral blood monocytes isolated from healthy donors. Since these particular TLR3 agonists have not been tested in the in vitro immunological systems, the TLR3 agonists give similar results to TLR7/8 agonists can be confirmed. LC is treated or left untreated with Poly-ICLC or Poly-ICR, then HPV can be added for 48 h. Cell surface molecules are measured by flow cytometry using fluorescent antibodies. Cell culture supernatants are tested for the presence of immune-stimulatory cytokines to determine whether LC have become activated and are secreting T cell-activating cytokines LC migration after exposure to HPV and Poly-ICLC or Poly-ICR are measured by in vitro migration through a transwell membrane. The capacity to induce HPV-specific T cells are measured by repeated in vitro immunization of naïve T cells with autologous LC incubated with Poly-ICLC or Poly-ICR agonists and HPV. The number of HPV-specific T cells induced with each treatment are quantified by an interferon gamma ELISpot assay.

LC's capacity to migrate and induction of inflammation after topical application of a TLR3 agonist on mouse ears can be used as an assay for bioactivity and toxicity. Either Poly-ICLC or Poly-ICR can be formulated into a cream-based vehicle which has been used safely applied to the cervix in humans without overt toxicity. The TLR3 agonist is painted on clean ears or skin of anesthetized mice for up to 4 h. At various time points, ears and skin are examined for signs of induration or inflammation. To test topical TLR3 agonist function on LC activation and migration, after 4 h, ears are removed, epidermal sheets separated, and numbers of LC that have migrated out of the tissue are determined. The contra lateral ear serves as a negative control. LC migration in the presence of HPV and a TLR3 agonist is also analyzed. HPV VLP is injected into the epidermal sheet, followed by application of the TLR3 agonist. At various time points, LC is isolated from epidermal sheets and analyzed for activation markers. These data indicate that if the topically applied TLR3 agonist is able to re-establish the migration capacity of HPV-infected LC in vivo. If so the data provides further evidence that HPV infected LC are gaining back the actual capacity to migrate towards T cells and potentially stimulate them.

The test of the reversal of the HPV immune escape mechanism through TLR3 agonist treatment can be induced in LC derived from cervical intraepithelial neoplasis (CIN) II/III patients. LC's derived from patients with CIN II/III capacity to produce cytokines and induce HPV-specific cytotoxic T cells can be determined. For feasibility reasons, experiments with human LC are performed using monocyte-derived LC instead of viable and naïve LC isolated from cervical tissue.

Cervical cancer is due to an infectious agent, HPV and there is a need for a topical agent feasible for use in industrial, developing and underdeveloped countries. Applicants contemplate that the methods and compositions described herein will simultaneously treat early cervical pre-cancerous lesions, aid in the generation of anti-HPV immunity, and break the cycle of HPV persistence and transmission. As HPV continues to replicate in its host, new viruses come into contact with LC reinforcing the immune escape mechanism. If, however, LC are activated with a topical TLR agonist, Applicants contemplate that LC will take up the viral particle, become activated and present viral capsid peptides to T cells, and activate T cells against HPV late protein antigens. T cell responses against the early viral proteins can then occur through cross-priming by uninhibited LC when virus-infected keratinocytes are killed. Applicants contemplate that women diagnosed with abnormal Pap smears would receive a cream-based TLR3 agonist for application on the cervix. This approach is a low-cost alternative to repeated screening or expensive surgical intervention with a high reasonable expectation of success because it targets the cause of cervical cancer development, namely HPV persistence and its related immune escape. Applicants further contemplate that generation of immunity against HPV will help clear the existing lesion and prevent future lesions from developing. The advantage of this approach over therapeutic vaccination with traditional vaccine platforms is that it may not be necessary to determine which HPV subtype should be targeted, which antigens should be targeted or the HLA status of the patient because the natural immune response will decide all those factors internally.

Unless otherwise defined, all technical and scientific terms used herein have the same meaning as commonly understood by one of ordinary skill in the art to which this invention belongs. All nucleotide sequences provided herein are presented in the 5′ to 3′ direction.

The inventions illustratively described herein may suitably be practiced in the absence of any element or elements, limitation or limitations, not specifically disclosed herein. Thus, for example, the terms “comprising”, “including,” containing”, etc. shall be read expansively and without limitation. Additionally, the terms and expressions employed herein have been used as terms of description and not of limitation, and there is no intention in the use of such terms and expressions of excluding any equivalents of the features shown and described or portions thereof, but it is recognized that various modifications are possible within the scope of the invention claimed.

Thus, it should be understood that although the present invention has been specifically disclosed by preferred embodiments and optional features, modification, improvement and variation of the inventions embodied therein herein disclosed may be resorted to by those skilled in the art, and that such modifications, improvements and variations are considered to be within the scope of this invention. The materials, methods, and examples provided here are representative of preferred embodiments, are exemplary, and are not intended as limitations on the scope of the invention.

The invention has been described broadly and generically herein. Each of the narrower species and subgeneric groupings falling within the generic disclosure also form part of the invention. This includes the generic description of the invention with a proviso or negative limitation removing any subject matter from the genus, regardless of whether or not the excised material is specifically recited herein.

In addition, where features or aspects of the invention are described in terms of Markush groups, those skilled in the art will recognize that the invention is also thereby described in terms of any individual member or subgroup of members of the Markush group.

All publications, patent applications, patents, and other references mentioned herein are expressly incorporated by reference in their entirety, to the same extent as if each were incorporated by reference individually. In case of conflict, the present specification, including definitions, will control.

It is to be understood that while the disclosure has been described in conjunction with the above embodiments, that the foregoing description and examples are intended to illustrate and not limit the scope of the disclosure. Other aspects, advantages and modifications within the scope of the disclosure will be apparent to those skilled in the art to which the disclosure pertains.

What is claimed is:

1. A method for activating a Langerhans cell (LC) exposed to a human papillomavirus (HPV), comprising contacting the LC with a toll-like receptor (TLR) agonist, thereby activating the LC.

2. A method for treating a human papillomavirus (HPV) infection in a subject, comprising administering to the subject an effective amount of a toll-like receptor (TLR) agonist, thereby treating the HPV infection in the subject.

3. A method for treating a pre-cancerous lesion induced by HPV infection in a subject, comprising administering to the subject an effective amount of a toll-like receptor (TLR) agonist, thereby treating the pre-cancerous lesion in the subject.

4. The method of any of claim 3, wherein the pre-cancerous lesion induced by HPV infection is a cervical, an anal, a vaginal, a vulvar, a penile, a tracheal, a laryngealor a head and neck pre-cancerous lesion

5. The method of any one of claims 1-3, wherein the TLR agonist is a TLR3 agonist, a TLR8 agonist, a TLR9 agonist, or combinations thereof.

6. The method of claim 5, wherein the TLR agonist is a TLR3 agonist.

7. The method of claim 5, wherein the TLR agonist is selected from the group of 3M-002 (CL075), or Resiquimod (R848).

8. The method of claim 5, wherein the TLR agonist is selected from the group of poly-IC, poly-ICLC, poly-ICR, CL264, Gardiquimod™, Loxoribine, CL075, CL097, and IC-31®.

9. The method of claim 2, further comprising administering to the subject an effective amount of an inflammatory agent, an analgesic, or an anti-human immunodeficiency virus (HIV) agent.

10. The method of claim 9, wherein the anti-HIV agent is selected from the group of nucleoside and nucleotide reverse transcriptase (RT) inhibitors; non-nucleoside reverse transcriptase inhibitors; protease inhibitors (PIs); viral absorption inhibitors; or viral coreceptor agonists.

11. The method of claim 9, wherein the analgesic is selected from the group of paracetamol, non-steroidal anti-inflammatory drug, COX-2 inhibitor, opiate or morphinomimetic.

12. The method of any of claim 1, 2 or 3, wherein the administration is vaginal, rectal, penile, oral, musosal, topical, or on skin surface.

13. A pharmaceutical formulation comprising a toll-like receptor (TLR) agonist and a pharmaceutically acceptable carrier.

14. The formulation of claim 13, further comprising an inflammatory agent, an analgesic, or an anti-human immunodeficiency virus (HIV) agent.

15. The formulation of claim 14, wherein the anti-HIV agent is selected from the group of nucleoside and nucleotide reverse transcriptase (RT) inhibitors; non-nucleoside reverse transcriptase inhibitors; protease inhibitors (PIs); viral absorption inhibitors; or viral coreceptor agonists.

16. A method for screening for a toll-like receptor (TLR) agonist suitable for activating a Langerhans cell (LC) exposed to a human papillomavirus (HPV), comprising contacting a candidate toll-like receptor (TLR) agonist with a test sample comprising a LC in the presence of a human papillomavirus (HPV), wherein a increased activation of the LC compared to a suitable control indicates that the candidate TLR agonist is suitable for activating LC exposed to a HPV.

17. The method of claim 16, wherein the suitable control is a TLR agonist selected from one or more of 3M-002 (CL075), Resiquimod (R848), poly-IC, poly-ICLC, poly-ICR, CL264, Gardiquimod™, Loxoribine, CL075, or CL097.
